SEQUENCE LISTING (27 sequences)

SEQ ID NO: 1 - protein, 24 aa, Homo sapiens
MAPRGFSCLLLLTSEIDLPVKRRA

SEQ ID NO: 2 - protein, 24 aa, Artificial
  an artificially synthesized polypeptide
MAPRGFSCLLLLTGEIDLPVKRRA

SEQ ID NO: 3 - protein, 24 aa, Artificial
  an artificially synthesized polypeptide
MAPRGFSALLLLTSEIDLPVKRRA

SEQ ID NO: 4 - protein, 24 aa, Artificial
  an artificially synthesized polypeptide
MAPRGFSCLLLLTSEIDLPVKRRA

SEQ ID NO: 5 - protein, 24 aa, Artificial
  an artificially synthesized polypeptide
MAPRGFSCLLLLTSEIDLPVKRRA

SEQ ID NO: 6 - protein, 24 aa, Artificial
  an artificially synthesized polypeptide
MAPRGFSCLLLLTSEIDLPVKRRA

SEQ ID NO: 7 - protein, 24 aa, Artificial
  an artificially synthesized polypeptide
MAPRGFSCLLLLTSEIDLPVKRRA

SEQ ID NO: 8 - protein, 24 aa, Artificial
  an artificially synthesized polypeptide
MAPRGFSCLLLLTSEIDLPVKRRA

SEQ ID NO: 9 - protein, 24 aa, Artificial
  an artificially synthesized polypeptide
MAPRGFSCLLLLTSEIDLPVKRRA

SEQ ID NO: 10 - protein, 24 aa, Artificial
  an artificially synthesized polypeptide
MAPAGASCLLLLTSEIDLPVKRRA

SEQ ID NO: 11 - protein, 17 aa, Artificial
  an artificially synthesized polypeptide
PAGASCLLLLTSEIDLP

SEQ ID NO: 12 - protein, 24 aa, Artificial
  an artificially synthesized polypeptide
MAPRGFACLLLLTSEIDLPVKRRA

SEQ ID NO: 13 - protein, 17 aa, Artificial
  an artificially synthesized polypeptide
PRGFACLLLLTSEIDLP

SEQ ID NO: 14 - protein, 25 aa, Artificial
  an artificially synthesized polypeptide
YMAPRGFSCLLLLTGEIDLPVKKKK

SEQ ID NO: 15 - protein, 32 aa, Artificial
  an artificially synthesized polypeptide
EFLIVIKSMAPRGFACLLLLTSEIDLPVKRRA

SEQ ID NO: 16 - protein, 32 aa, Artificial
  an artificially synthesized polypeptide
EFLIVIKSMAPRGFSCLLLLTGEIDLPVKKKK

SEQ ID NO: 17 - protein, 32 aa, Artificial
  an artificially synthesized polypeptide
EFLIVIKSMAPRGFSCLLLLTSEIDLPVKRRA

SEQ ID NO: 18 - protein, 32 aa, Artificial
  an artificially synthesized polypeptide
EFLIVIKSMAPRGFSALLLLTSEIDLPVKRRA

SEQ ID NO: 19 - protein, 32 aa, Artificial
  an artificially synthesized polypeptide
EFLIVIKSMAPRGFSCLLLLTGEIDLPVKRRA

SEQ ID NO: 20 - protein, 8 aa, Artificial
  an artificially synthesized polypeptide
EFLIVIKS

SEQ ID NO: 21 - protein, 24 aa, Artificial
  an artificially synthesized polypeptide
MAPAGASCLLLLTGEIDLPVKRRA

SEQ ID NO: 22 - protein, 32 aa, Artificial
  an artificially synthesized polypeptide
EFLIVIKSMAPAGASCLLLLTGEIDLPVKRRA

SEQ ID NO: 23 - protein, 17 aa, Artificial
  an artificially synthesized polypeptide
PRGFSCLLLLTGEIDLP

SEQ ID NO: 24 - protein, 7 aa, Artificial
  an artificially synthesized polypeptide
EFLIVKS

SEQ ID NO: 25 - protein, 17 aa, Artificial
  an artificially synthesized polypeptide
PRGFSCLLLLTSEIDLP

SEQ ID NO: 26 - DNA, 72 bp, Homo sapiens
  CDS (1)..(72)
atggctccacgagggttcagctgtctcttacttttaaccagtgaaattgacctgcccgtgaagaggcgggca

SEQ ID NO: 27 - protein, 24 aa, Homo sapiens
MAPRGFSCLLLLTSEIDLPVKRRA

(19) United States
(12) Patent Application Publication
(10) Pub. No.: US 2005/0233413 A1
(43) Pub. Date: Oct. 20, 2005

(54) Neuroprotective polypeptides and methods of use

(76) Inventors: Ikuo Nishimoto, Ichikawa-shi (JP); Tomo Nishimoto, Ichikawa-shi (JP)

Correspondence Address: FISH & RICHARDSON PC, P.O. BOX 1022, MINNEAPOLIS, MN, 55440-1022, US

(21) Appl. No.: 10/514,653

(22) PCT Filed: May 16, 2003
(86) PCT No.: PCT/JP03/06139

§ 371 Date: Jun. 21, 2005

Related U.S. Application Data
(60) Provisional application No. 60/380,958, filed on May 16, 2002.

(51) Int. Cl. A61K 38/10; C07K 7/08; C07H 21/04

(52) U.S. Cl. 435/69.1; 514/14; 530/326; 435/320.1; 435/325; 536/23.2; 514/7

(57) ABSTRACT

Humanin (HN), which has the sequence: Met-Ala-Pro-Arg-Gly-Phe-Ser-Cys-Leu-Leu-Leu-Leu-Thr-Ser-Glu-Ile-Asp-Leu-Pro-Val-Lys-Arg-Arg-Ala, is a polypeptide that was identified by screening for molecules that suppress neuronal cell death induced by a gene mutant associated with an familial Alzheimer's disease. The present application provides polypeptides derived from Humanin, which contain one or more D-amino acids or phosphorylated amino acids, or amino acids that form a multimer. The HN derivatives are useful in protecting neuronal cells from cytotoxicity related to neurodegenerative diseases. With higher potency and resulting lower effective amounts, HN derivatives represent better therapeutic agents than the naturally occurring form of RN.

### PRIORITY INFORMATION

This application claims priority from U.S. Provisional Application Ser. No. 60/380,958, filed on May 16, 2002, which is incorporated herein by reference in its entirety.

### TECHNICAL FIELD

This invention relates to new polypeptides and their use for neuroprotection, e.g., to treat neurodegenerative disorders such as Alzheimer's disease.

### BACKGROUND ART

Alzheimer's disease (AD) is the most prevalent neurodegenerative disease associated with progressive dementia. No fundamental therapy for this disease has so far been developed. As brain atrophy is the central abnormality in AD, pathological mechanisms leading to neuronal loss must be understood in order to establish future curative therapy for AD.

Three mutant genes are known to cause early-onset familial AD (FAD): amyloid precursor protein mutants, presenilin (PS)1 mutants, and PS2 mutants [1]. All examined FAD mutants cause or enhance cytotoxicity when they are expressed in neuronal cells. Therefore, molecules that suppress the cytotoxicity may be useful in treating AD and other neurodegenerative diseases.

Humanin (WN), which has the sequence: Met-Ala-Pro-Arg-Gly-Phe-Ser-Cys-Leu-Leu-Leu-Leu-Thr-Ser-Glu-Ile-Asp-Leu-Pro-Val-Lys-Arg-Arg-Ala (SEQ ID NO: 1), is a polypeptide that was identified by screening for molecules that suppress neuronal cell death induced by an FAD gene mutant. HN has been show to protect neural cells against cell death. An HN derivative, which has a Gly substitution of Ser¹⁴ of HN (symbolized as S14G), markedly potentiates the death-suppressing function of HN. While HN exerts full neuroprotection at low μM levels, (S14G)HN, also referred to as “HNG” for short (SEQ ID NO: 2), does so at low nM concentrations.

### DISCLOSURE OF THE INVENTION

The invention is based, at least in part, on the discovery that by making certain modifications in the wild-type 24-residue Humanin (HN) polypeptide, one can obtain HN derivatives can have significantly higher neuroprotective activity than HN. These HN derivatives are based on the recognition that D-isomers of HN and multimers, e.g., dimers, of HN have higher neuroprotective activity. Based on these discoveries, the invention provides new HN derivatives and methods of using them to protect neuronal cells against the otherwise cytotoxic effects of neurodegenerative disorders, such as Alzheirner's Disease.

In general, in one embodiment, the invention features purified HN derivatives that protect neuronal cells from cytotoxicity, wherein the derivatives contain at least one D-amino acid, e.g., at the position of Ser¹⁴ of Humanin. For example, the D-amino acid can be D-Serine or D-Proline.

In another embodiment, the invention includes HN derivatives that protect neuronal cells from cytotoxicity, wherein the derivatives are capable of forming a multimer, e.g., wherein the derivatives contain one or more amino acids that are capable of binding to each other. For example, the sequence of the amino acids can be EFLIVIKS (SEQ ID NO: 20) or EFLIVKS (SEQ ID NO: 24).

As used herein, “a derivative of Humanin” means a polypeptide in which one or more amino acids are altered from authentic Humanin polypeptide (SEQ ID NO: 1). The HN derivatives can be selected from the group consisting of the polypeptides: Humanin with S14P (SEQ ID NO: 4), P-S7 HN (SEQ ID NO: 5), P-S7/14 HN (SEQ ID NO: 6), (D-Ser¹⁴)HN (SEQ ID NO: 7), (D-Ser⁷)HN (SEQ ID NO: 8), (D-Ser^{7/14})HN (SEQ ID NO: 9), AGA-(D-Ser¹⁴)HN (SEQ ID NO: 10), AGA-(D-Ser¹⁴)HN17 (SEQ ID NO: 11), EFLIVIKS-HNG (SEQ ID NO: 19), EFLIVIKS-HNA (SEQ ID NO: 18), EFLIVIKS-HN (SEQ ID NO: 17), EFLIVIKS-HNG-KKK (SEQ ID NO: 16), EFLIVIKS-(S7A)HN (SEQ ID NO: 15), and EFLIVIKS-AGA-HNG (SEQ ID NO: 22), and chimeric combinations thereof. As used herein, the term “S14P” means that the S (serine) at location 14 in the wild-type HN has been replaced with P (proline). The same convention applies for other substitutions (e.g., S7A). “D-Ser⁷” means that the Serine at location 7 has been switched (racemized) from a normal L-isomer to the D-isomer. “AGA-HN” is a shorthand name of the HN derivative in which the Arg⁴ and Phe⁶ amino acids are substituted with Alanine to form R4A/F6A-HN (this is named for the AGA triplet at locations 4, 5, and 6 in the HN derivative. “HN17” is a truncated form of HN that includes 17 amino acids from Pro³ to Pro¹⁹.

In other aspects, the invention includes nucleic acids that encode the new HN derivatives, and methods for protecting neuronal cells from cytotoxicity by contacting the cells with an effective amount of the new HN derivatives. The invention further includes pharmaceutical compositions that include the derivatives and one or more pharmaceutically acceptable carriers. In another aspect, the invention features methods for treating an individual suffering from or suspected of having a neurodegenerative disorder, by administrating to the individual an amount of the new HN derivatives effective to treat the disorder. For example, the disorder can be Alzheimer's disease.

As used herein, both “protein” and “polypeptide” mean any chain of amino acid residues, regardless of length or post-translational modification (e.g., glycosylation or phosphorylation). Modification also includes acetylation; acylation; ADP ribosylation; amidation; covalent bonding with flavin, nucleotide, nucleotide derivative, lipid, lipid derivative, or phosphatidyl inositol, and such; cross link formation; cyclization; disulfide bond formation; demethylation; pyroglutamylation; γ-carboxylation; hydroxylation; iodization; methylation; myristoylation; oxidation; ubiquitination; and so on. The HN derivatives useful in the invention are referred to as “purified” or “substantially pure,” meaning that a composition containing the polypeptide is at least 60% by weight (dry weight) the polypeptide of interest, e.g., a HN derivative. Preferably, the polypeptide composition is at least 75%, more preferably at least 90%, and most preferably at least 99%, by weight, the polypeptide of interest. Purity can be measured by any appropriate standard method, e.g., column chromatography, polyacrylamide gel electrophoresis, or HPLC analysis.

An effective amount of an HN derivative composition is an amount that provides a measurable reduction in symptoms of a neurodegenerative disorder.

In the case of polypeptide sequences that are less than 100% identical to a reference sequence, the non-identical positions can be conservative substitutions for the reference sequence or substitutions of non-essential amino acids. A “non-essential” amino acid residue is a residue that can be altered from the wild-type sequence of HN (e.g., the sequence of SEQ ID NO: 1) without abolishing or substantially altering the neuroprotectant activity, whereas an “essential” amino acid residue results in such a change.

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below. All publications, patent applications, patents, and other references mentioned herein are incorporated by reference in their entirety. In case of conflict, the present specification, including definitions, will control. In addition, the materials, methods, and examples are illustrative only and not intended to be limiting.

The HN derivatives are useful in protecting neuronal cells from cytotoxicity related to neurodegenerative diseases. With higher potency and resulting lower effective amounts, HN derivatives represent better therapeutic agents than the naturally occurring form of HN. The lower effective amount of the peptides can minimize toxicity or side effects that might be associated with the HN derivatives. Also, the lower effective amount renders the manufacturing (e.g., genetic engineering or a chemical synthesis) and purifying of these peptides economically more practical. In addition, the HN derivatives provide the pharmaceutical industry an effective tool to study the pathological mechanism of neurodegenerative diseases and to develop more drugs.

Other features and advantages of the invention will be apparent from the following detailed description, and from the claims.

### BRIEF DESCRIPTION OF THE DRAWINGS

FIG. 1 shows representative cellular Calcein fluorescence microscopic photographs representing results of neuronal cytotoxicity study in the presence or absence of D-Ser HN peptides at indicated concentrations. Effects of D-Ser HN peptides on neuronal cytotoxicity by Aβ1-43 or FAD genes are shown.

Primary cultured neurons were treated with 25 μM Aβ1-43 and cultured in the presence or absence of indicated concentrations of (D-Ser¹⁴)HN or (D-Ser⁷)HN. Seventy-two hr after the onset of Aβ treatment, alive neurons were stained with Calcein-AM. The representative microscopic views of cellular Calcein fluorescence are indicated.

FIG. 2 shows graphs representing results of neuronal cytotoxicity study in the presence or absence of D-Ser HN peptides at indicated concentrations. Effects of D-Ser HN peptides on neuronal cytotoxicity by Aβ1-43 or FAD genes are shown.

Upper (A) and lower (B) left panels: Primary neurons (2.5×10⁴ cells/well) were treated with 25 μM Aβ1-43 and cultured in the presence or absence (−) of increasing concentrations of (D-Ser¹⁴)HN or (D-Ser⁷)N. Cell viability was measured 72 hr after Aβ treatment by Calcein fluorescence assay (upper panel) and WST-8 absorbance assay (lower panel), respectively.

Other panels (C-F): F11 cells were transfected with either FAD gene (V642I-APP cDNA, NL-APP cDNA, M146L-PS1 cDNA, N141I-PS2 cDNA) and cultured in the presence or absence (−) of various concentrations (10 pM, 100 pM, 1 nM, 10 nM, 100 nM, 1 μM, 10 μM, 100 μM (from left to right) of (D-Ser¹⁴)HN or (D-Ser⁷)HN as indicated. Cell mortality was measured 72 hr after transfection by Trypan blue exclusion assay. In each experiment, cells were incubated with 10 nM HNG (HNG) as a positive control after transfection. As a negative control (No T) in each experiment, cells were exposed to the transfection procedure without plasmids or exposed to treatment without Aβ, and cultured with the medium for 72 hr. As another control, cells were transfected with an empty vector (vec) and cultured with the medium for 72 hr. Unless otherwise specified, values presented in the Examples indicate means±S.D. of at least three independent experiments.

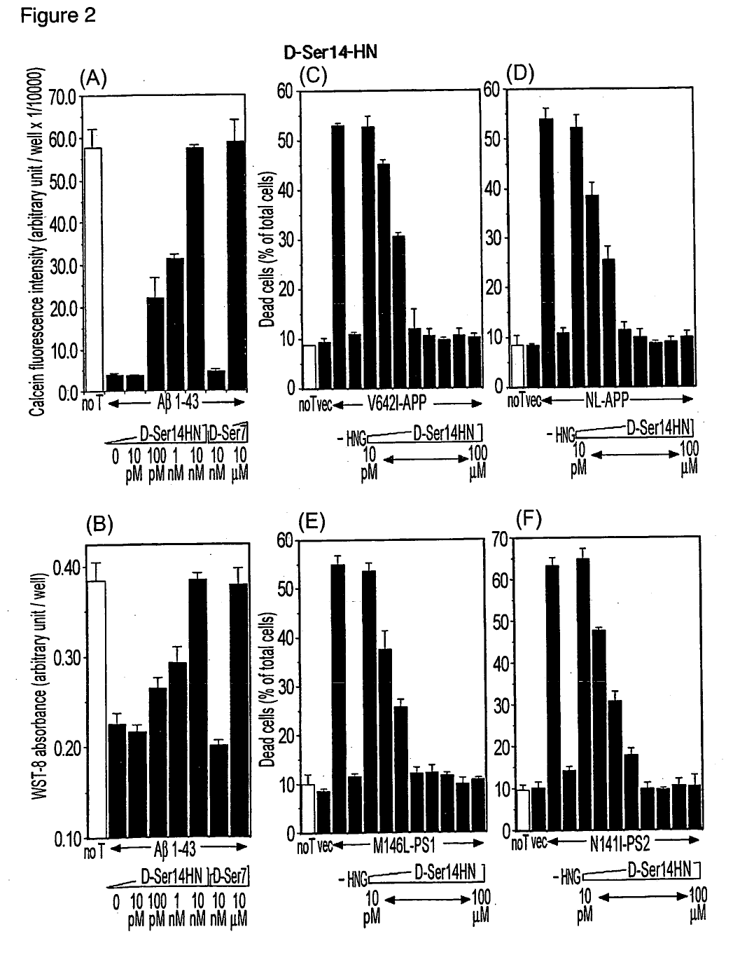

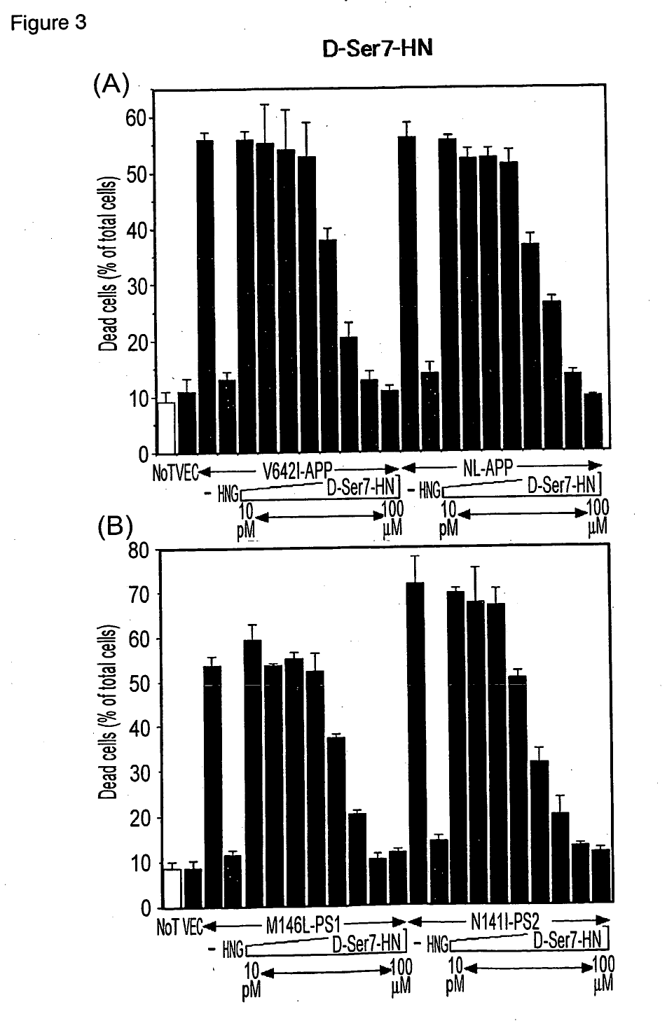

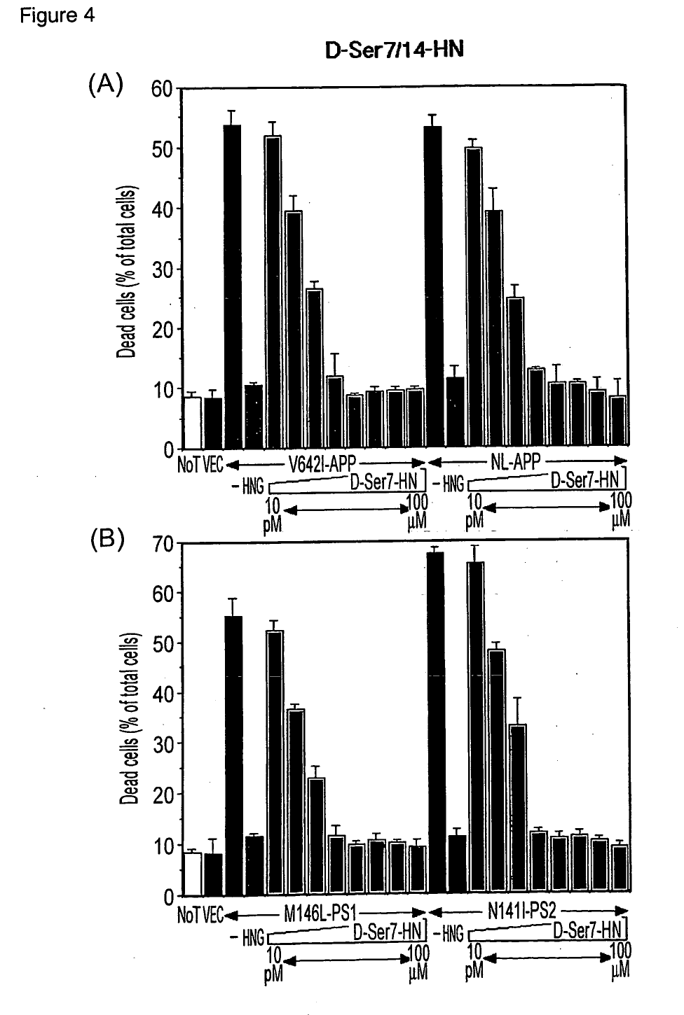

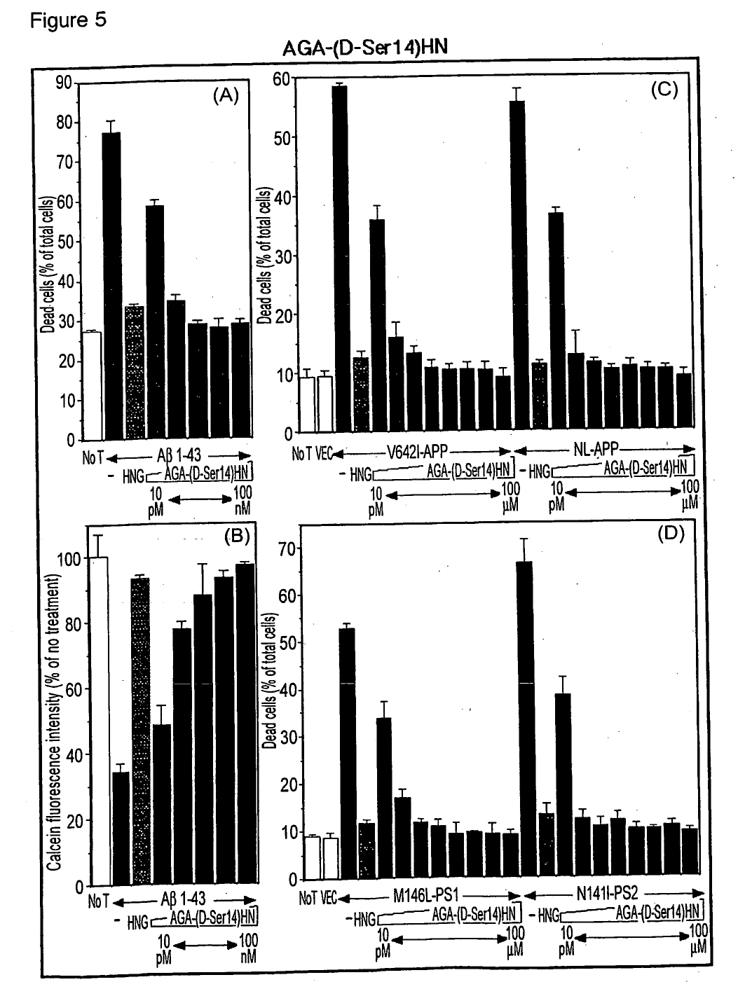

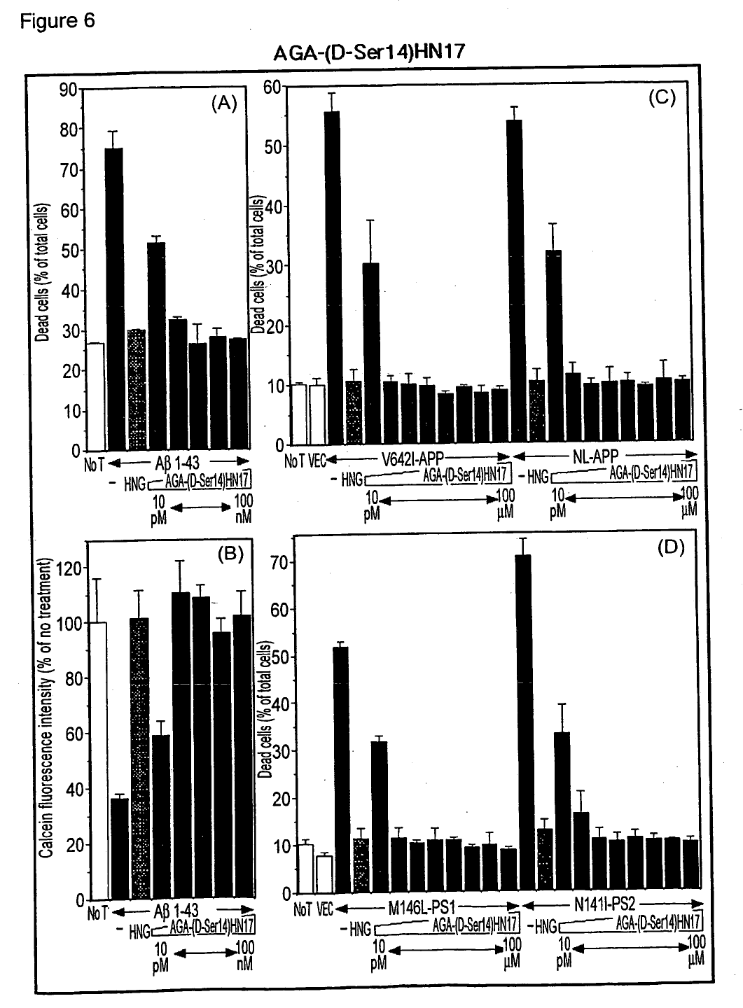

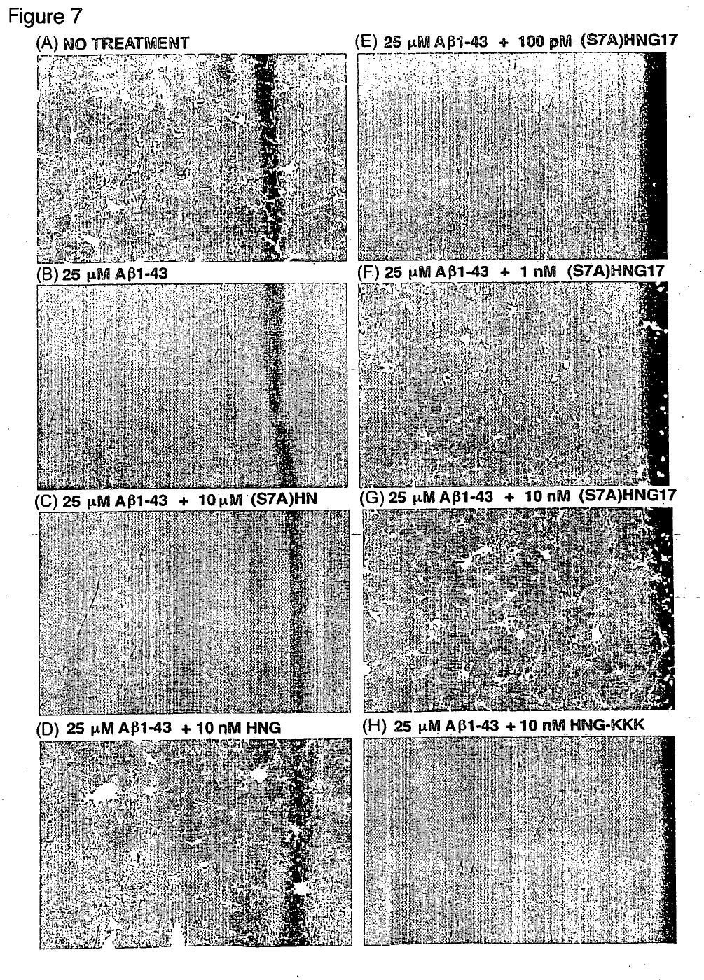

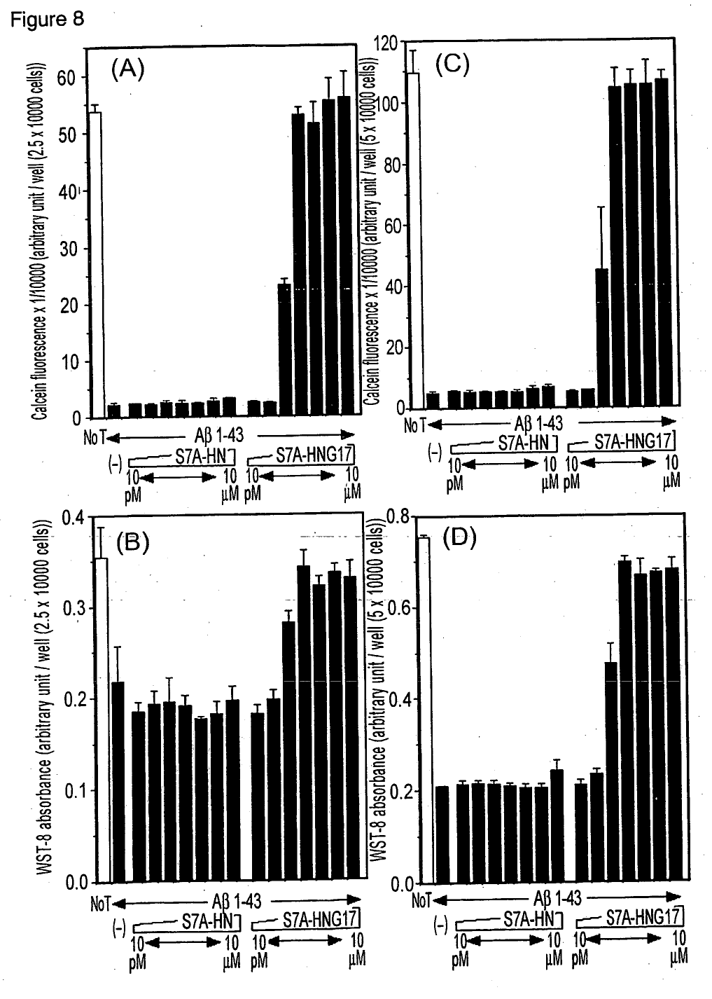

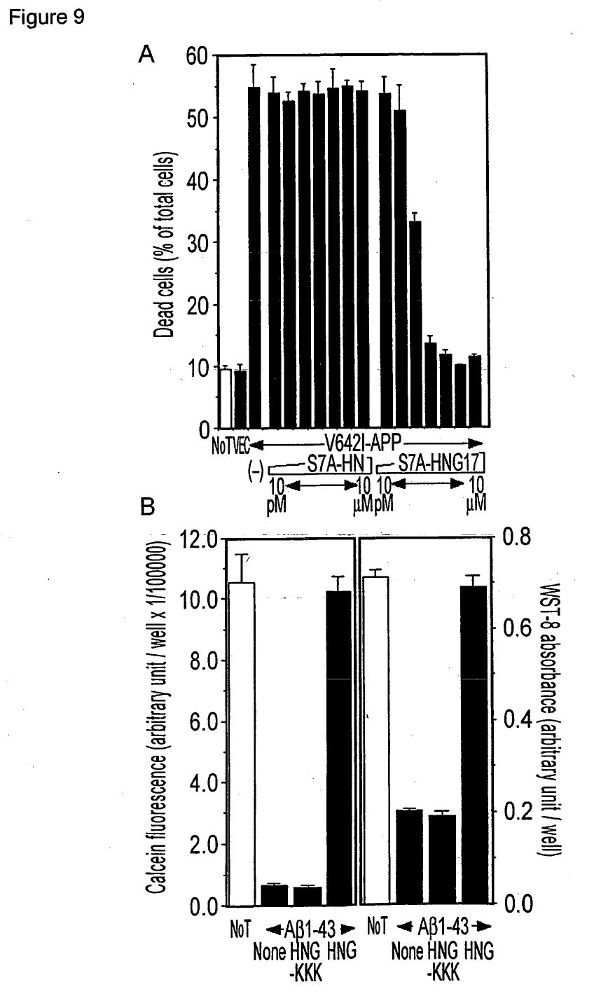

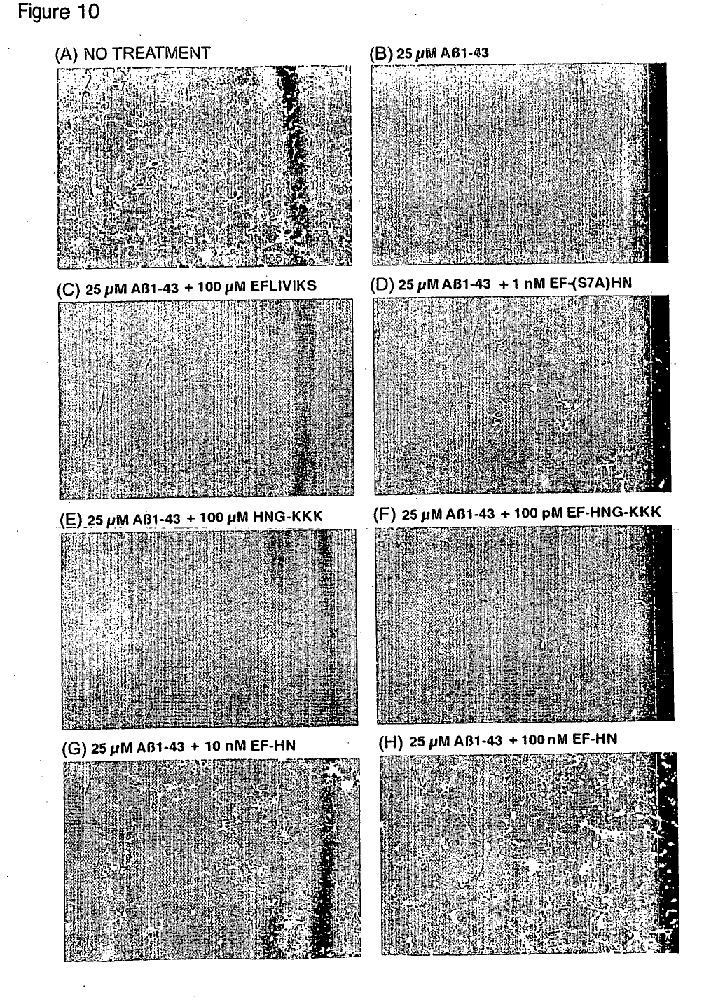

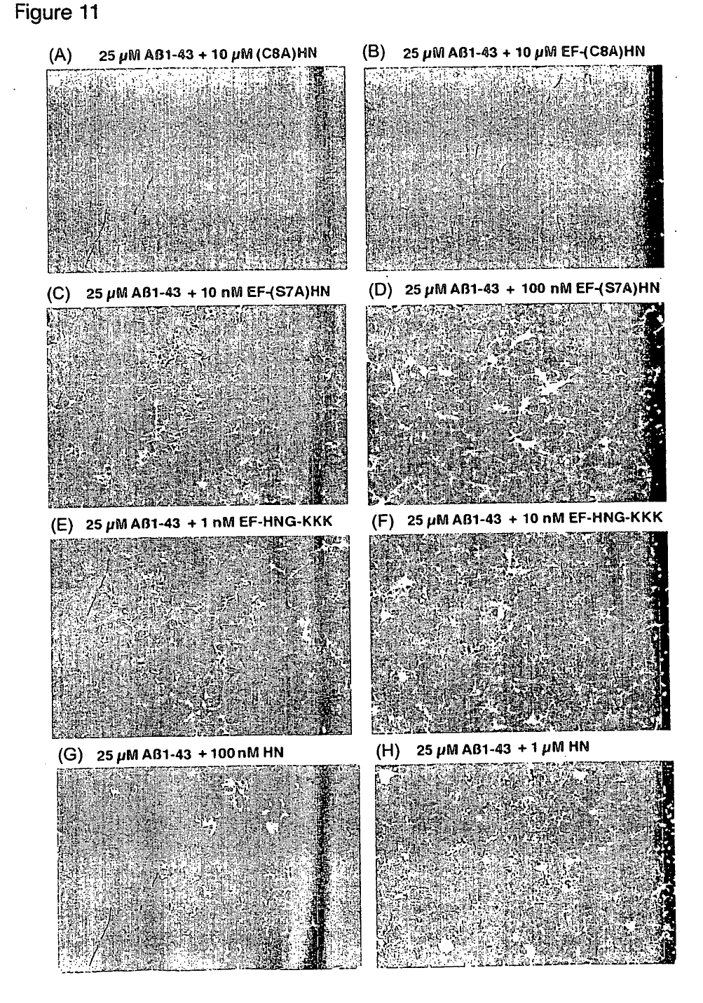

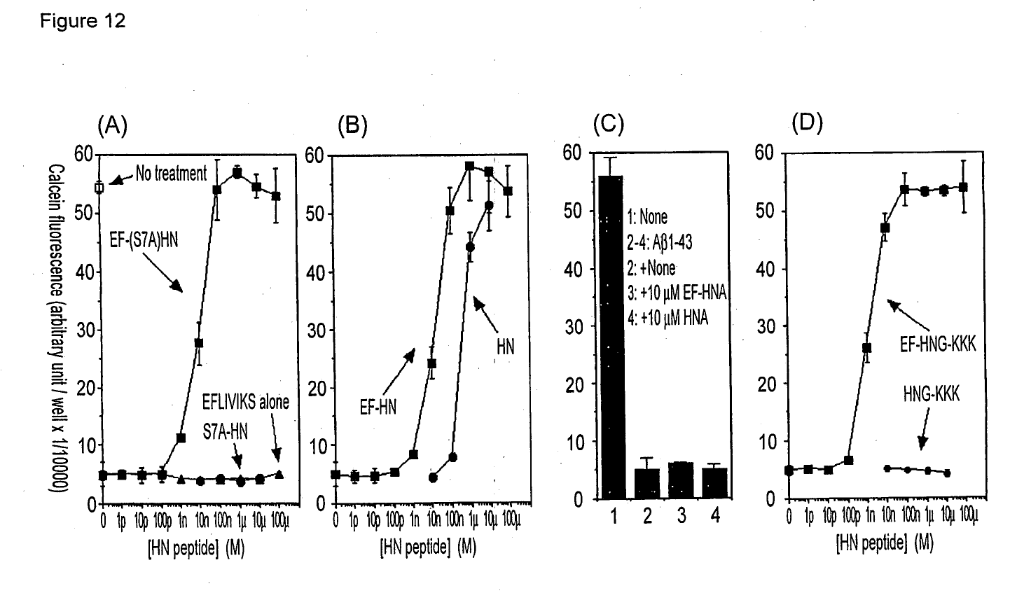

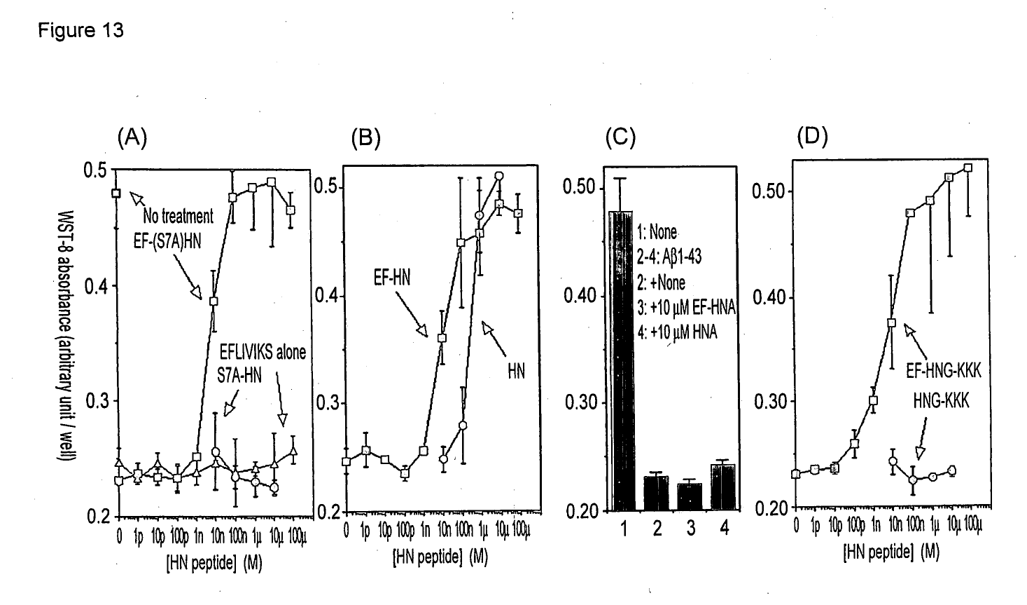

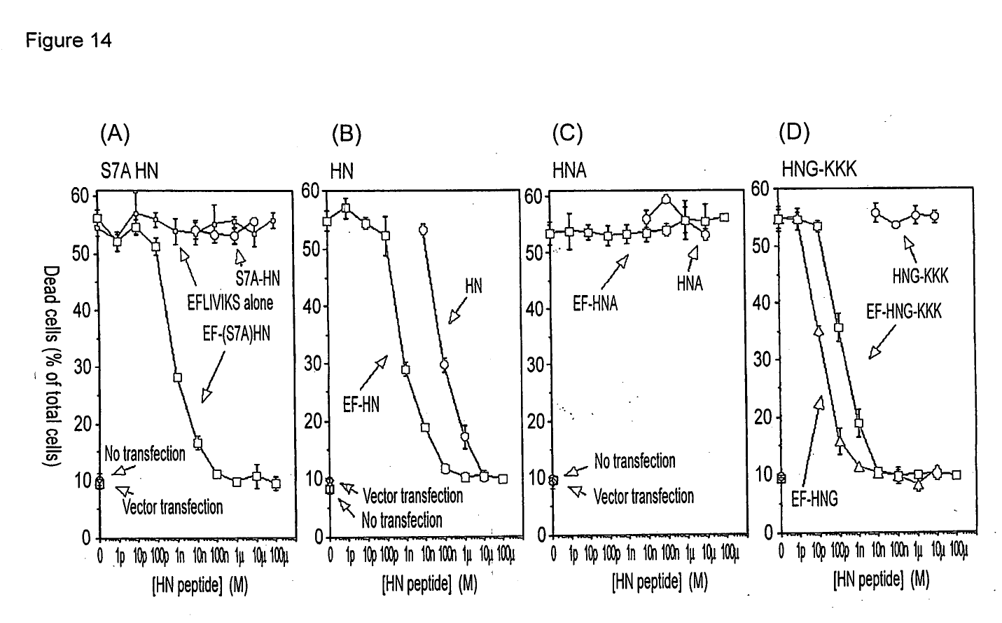

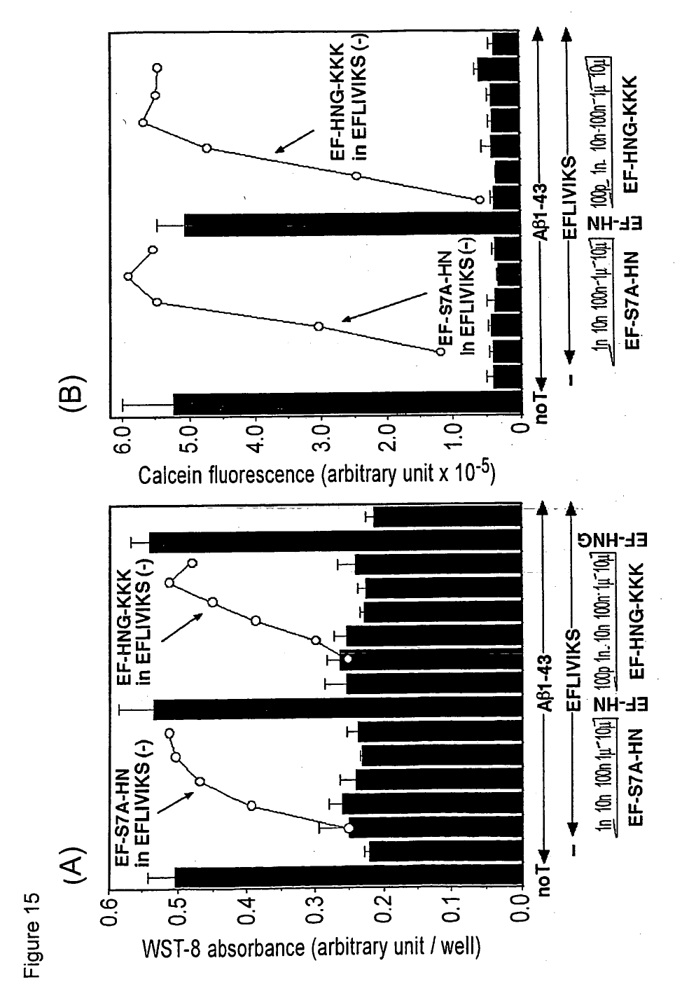

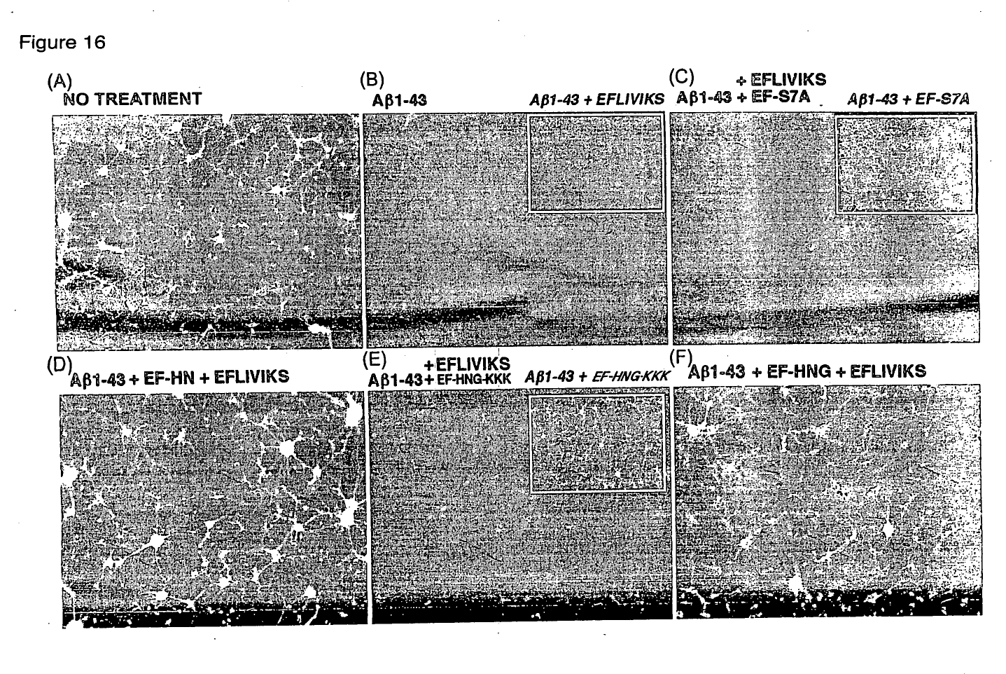

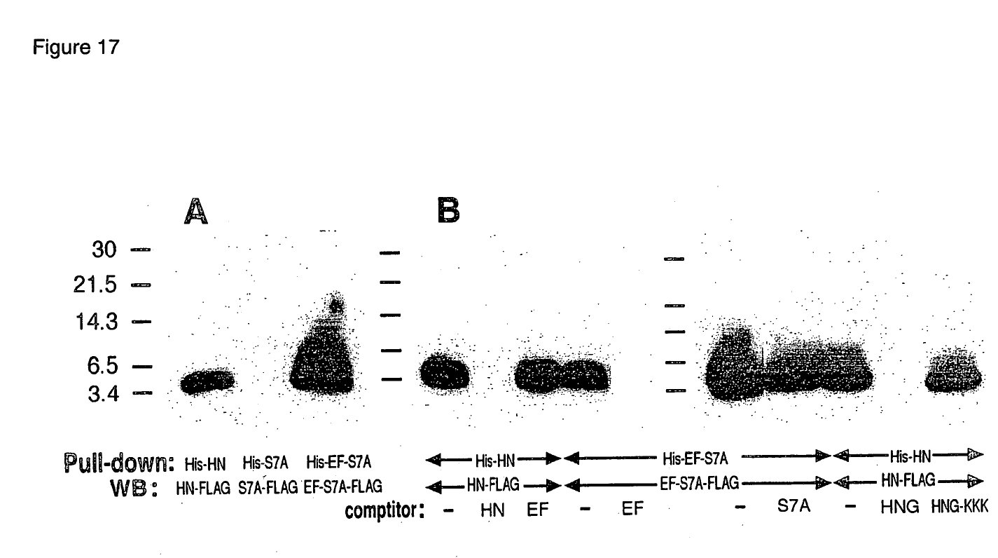

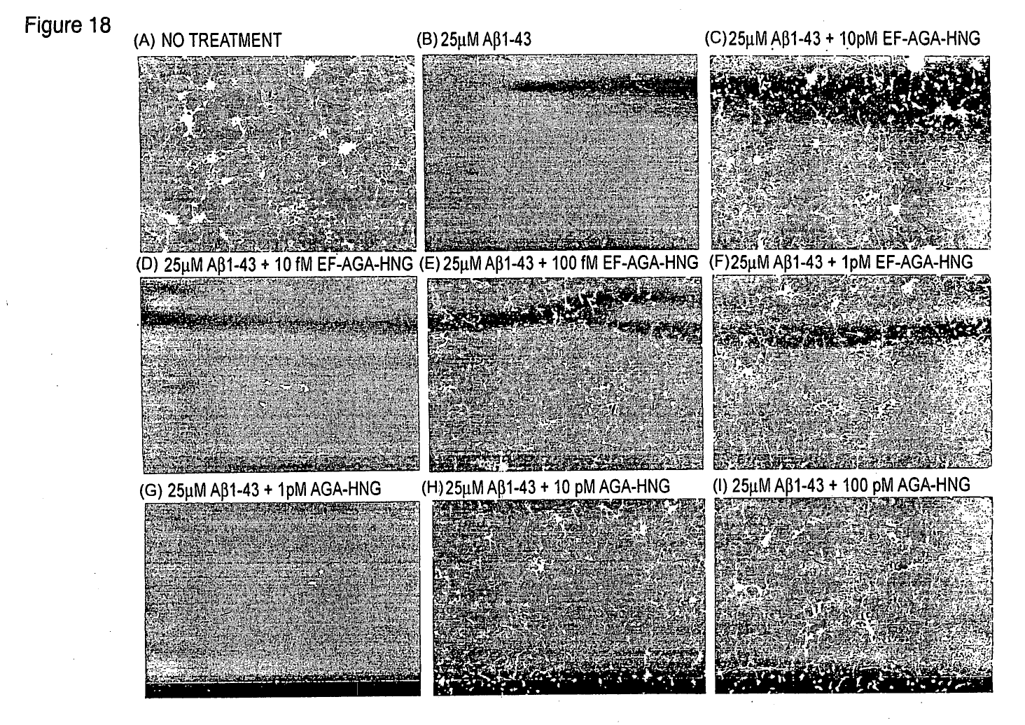

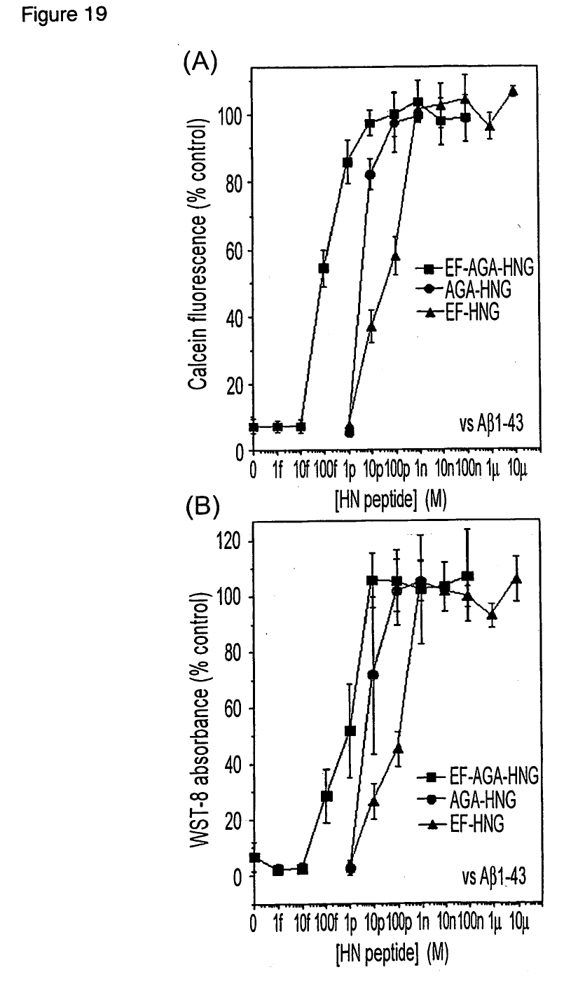

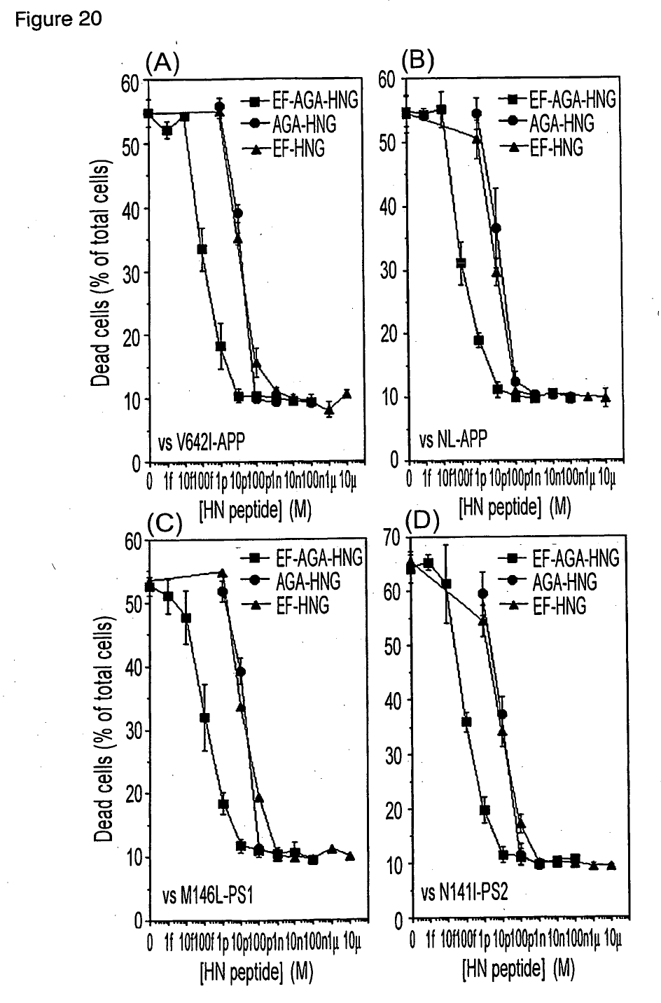

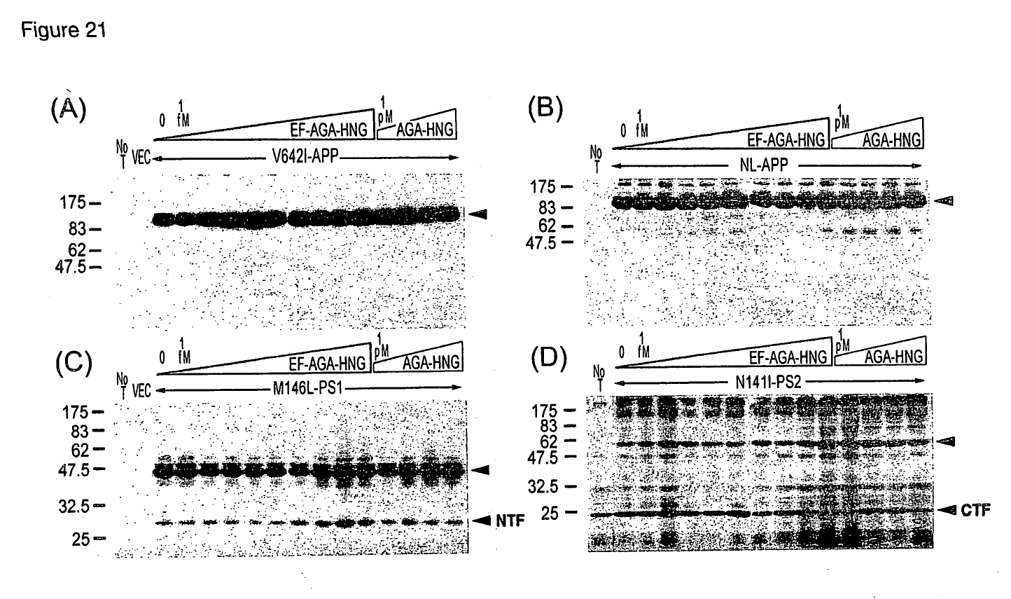

FIG. 3 shows graphs representing results of neuronal cytotoxicity study in the presence or absence of D-Ser HN peptides at indicated concentrations. F11 cells were transfected with either FAD gene (V642I-APP cDNA, NL-APP cDNA, M146L-PS1 cDNA, N141I-PS2 cDNA) and cultured in the presence or absence (−) of various concentrations (10 pM, 100 pM, 1 nM, 10 nM, 100 nM, 1 μM, 10 μM, 100 μM (from left to right) of (D-Ser¹⁴)HN or (D-Ser⁷)HN as indicated. Cell mortality was measured as same as in FIG. 2.

FIG. 4 shows graphs representing results of neuronal cytotoxicity study in the presence or absence of D-Ser HN peptides at indicated concentrations. F11 cells were transfected with either FAD gene (V642I-APP cDNA, NL-APP cDNA, M146L-PS1 cDNA, N141I-PS2 cDNA) and cultured in the presence or absence (−) of various concentrations (10 pM, 100 pM, 1 nM, 10 nM, 100 nM, 1 μM, 10 μM, 100 μM (from left to right) of (D-Ser^{7/14})HN as indicated. Cell mortality was measured as same as in FIG. 2.

FIG. 5 shows graphs representing results of neuronal cytotoxicity study in the presence or absence of AGA-(D-Ser¹⁴)HN at indicated concentrations. Effects of AGA-(D-Ser¹⁴)HN on neuronal cytotoxicity by Aβ1-43 or FAD genes are shown.

Upper (A) and lower (B) left subpanels: Primary cultured neurons were treated with 25 μM Aβ1-43 and cultured in the presence or absence (−) of various concentrations (10 pM, 100 pM, 1 nM, 10 nM, 100 nM from left to right) of AGA-(D-Ser¹⁴)HN. Both cell mortality and viability were measured 72 hr after Aβ treatment by Trypan blue exclusion assay (panels with % dead cells axes) and Calcein assay (panels with Calcein fluorescence intensity axes), respectively. The Calcein experiments were performed with neurons seeded in 24-well plates (see Examples). The same experiments with neurons seeded in 96-well plates yielded similar results. The value in the Calcein experiments indicates the percentage of the “no treatment” values.

Upper (C) and lower (D) right subpanels: F11 cells were transfected with either FAD gene (V642I-APP cDNA, NL-APP cDNA, M146L-PS1 cDNA, N141I-PS2 cDNA) and cultured in the presence or absence (−) of various concentrations (10 pM, 100 pM, 1 nM, 10 nM, 100 nM, 1 μM, 10 μM, 100 μM (from left to right) of AGA-(D-Ser¹⁴)HN. Cell mortality was measured 72 hr after transfection by Trypan blue exclusion assay. In each experiment, cells were incubated with 10 nM HNG as a positive control after transfection or Aβ treatment. As a negative control (No T) in each experiment, cells were exposed to the transfection procedure without plasmids or exposed to no treatment with Aβ, and cultured with the medium for 72 hr. As another control, cells were transfected with an empty vector (vec) and cultured with the medium for 72 hr.

FIG. 6 shows graphs representing results of neuronal cytotoxicity study in the presence or absence of AGA-(D-Ser¹⁴)HN17 at indicated concentrations. Effects of AGA-(D-Ser¹⁴)HN17 on neuronal cytotoxicity by Aβ1-43 or FAD genes were estimated as same as in FIG. 5.

FIG. 7 shows representative cellular Calcein fluorescence microscopic photographs representing results of neuronal cytotoxicity study in the presence or absence of (S7A)HN, (S7A)HNG17, HNG or HNG-KKK at indicated concentrations. Effects on neurocytotoxicity by Aβ1-43 are shown.

Primary cultured neurons were treated with 25 μM Aβ1-43 and cultured in the presence or absence of indicated concentrations of (S7A)HNG17, (S7A)HN, HNG, or HNG-KKK. Seventy-two hr after the onset of Aβ treatment, alive neurons were stained with Calcein-AM. The representative views of cellular Calcein fluorescence are indicated.

FIG. 8 shows graphs representing results of neuronal cytotoxicity study in the presence or absence of (S7A)HN or (S7A)HNG17 at indicated concentrations. Effects on neurocytotoxicity by Aβ1-43 are shown.

Primary neurons [cultured at 2.5×10⁴ cells/well (A, B) or 5×10⁴ cells/well (C, D)] were treated with 25 μM Aβ1-43 and cultured in the presence or absence (−) of various concentrations (10 pM, 100 pM, 1 nM, 10 nM, 100 nM, 1 μM, 10 μM from left to right) of (S7A)HN or (S7A)HNG17. Cell mortality was measured 72 hr after Aβ treatment by Calcein assay (B, D) or WST-8 assay (C, E). As a negative control (no T) in each experiment, cells were exposed to no treatment with Aβ, and cultured with the medium for 72 hr.

FIG. 9 shows graphs representing results of neuronal cytotoxicity study in the presence or absence of (S7A)HN, (S7A)HNG17, HNG or HNG-KKK at indicated concentrations. Effects on neurocytotoxicity by V642I-APP or Aβ1-43 are shown.

A: F11 cells were transfected with or without (no T) V642I-APP cDNA and cultured in the presence or absence (−) of various concentrations (10 pM, 100 pM, 1 nM, 10 nM, 100 nM, 1 μM, 10 μM from left to right) of (S7A)HN or (S7A)HNG17. Cell mortality was measured 72 hr after transfection by Trypan blue exclusion assay. As another control, cells were transfected with an empty vector (vec) and cultured with the medium for 72 hr.

B: Effect of HNG-KKK on neuronal death by Aβ1-43. Primary cultured neurons were treated with 25 μM Aβ1-43 and cultured in the presence or absence of 10 mM HNG-KKK or HNG. Cell viability was measured 72 hr after Aβ treatment by WST-8 assay (right subpanel) and Calcein assay (left subpanel), respectively. As a negative control (no T) in each experiment, cells were exposed to no treatment with Aβ, and cultured with the medium for 72 hr.

FIG. 10 shows representative cellular Calcein fluorescence microscopic photographs representing results of neuronal cytotoxicity study in the presence or absence of EFLIVIKS, EF-(S7A)HN, HNG-KKK, EF-HNG-KKK, or EF-HN at indicated concentrations.

Primary cultured neurons were treated with 25 μM Aβ1-43 and cultured in the presence or absence of indicated concentrations of EFLIVIKS, EF-(S7A)HN, HNG-KKK, EF-HNG-KKK, or EF-HN. Seventy-two hr after the onset of Aβ treatment, alive neurons were stained with Calcein-AM. The representative views of cellular Calcein fluorescence are indicated.

FIG. 11 shows representative cellular Calcein fluorescence microscopic photographs representing results of neuronal cytotoxicity study in the presence or absence of (C8A)HN(HNA), EF-(C8A)HN, EF-(S7A)HN, HNG-KKK, or HN at indicated concentrations.

Primary cultured neurons were treated with 25 μM Aβ1-43 and cultured in the presence or absence of indicated concentrations of EF-(S7A)HN, EF-HNG-KKK, EF-(C8A)HN, (C8A)HN(HNA), or HN. Seventy-two hr after the onset of Aβ treatment, alive neurons were stained with Calcein-AM. The representative views of cellular Calcein fluorescence are indicated.

FIG. 12 shows graphs representing results of neuronal cytotoxicity study in the presence or absence of (C8A)HN(HNA), EF-(C8A)HN, EFLIVIKS, EF-(S7A)HN, HNG-KKK, EF-HNG-KKK, EF-HN, or HN at indicated concentrations.

Primary cultured neurons were treated with 25 μM Aβ1-43 and cultured in the presence of increasing concentrations of EFLIVIKS-fused HN peptide or corresponding non-fused HN peptide. Cell viability was measured 72 hr after Aβ treatment by Calcein assay. As a negative control (no treatment), cells were exposed to no treatment with Aβ1-43 and cultured with the medium for 72 hr. The experiments using EF-HNA and HNA were performed with 10 μM peptides. Neurons were treated with 25 μM Aβ1-43 in the presence or absence of 10 μM EF-HNA or 10 μM HNA.

FIG. 13 shows graphs representing results of neuronal cytotoxicity study in the presence or absence of (C8A)HN(HNA), EF-(C8A)HN, EFLIVIKS, EF-(S7A)HN, HNG-KKK, EF-HNG-KKK, EF-HN, or HN at indicated concentrations.

Primary cultured neurons were treated with 25 μM Aβ1-43 and cultured in the presence of increasing concentrations of EFLIVIKS-fused HN peptide or corresponding non-fused HN peptide. Cell viability was measured 72 hr after Aβ treatment by WST-8 assay. As a negative control (no treatment), cells were exposed to no treatment with Aβ1-43 and cultured with the medium for 72 hr. The experiments using EF-HNA and HNA were performed with 10 μM peptides. Neurons were treated with 25 μM Aβ1-43 in the presence or absence of 10 μM EF-HNA or 10 μM HNA. In the right panel, the value of S.D. for the WST-8 cell viability in the presence of 100 nM EF-HNG-KKK was 0.149.

FIG. 14 shows graphs representing results of neuronal cytotoxicity study in the presence or absence of (C8A)HN(HNA), EF-(C8A)HN, EFLIVIKS, EF-(S7A)HN, HNG-KKK, EF-HNG-KKK, EF-HN, or HN at indicated concentrations.

F11 cells were transfected with V642I-APP cDNA and cultured in the presence of increasing concentrations of EFLIVIKS-fused HN peptide or corresponding non-fused HN peptide. Cell mortality was measured 72 hr after transfection by Trypan blue exclusion assay. As negative controls, cells were exposed to the transfection procedure without plasmids (no transfection) or transfected with an empty vector (vec transfection), and cultured with the medium for 72 hr. In the panel in the bottom right corner, the dose-response curve for the cytoprotective effect of EF-HNG, shown in FIG. 7B, is superimposed to ease comparison with the curve for the cytoprotective effect of EF-HNG-KKK.

FIG. 15 shows graphs representing blocking effect of EFLIVIKS on the neuroprotective action of EF-(S7A)HN, EF-HNG-KKK, EF-HN or EF-HNG.

Primary cultured neurons were treated with 25 μM Aβ1-43 and cultured with indicated increasing concentrations of EF-(S7A)HN (EF-S7A-HN) or EF-HNG-KKK (EF-HNG-KKK) in the presence of 100 μM EFLIVIKS. Seventy-two hr after the onset of Aβ treatment, viability of neurons was quantified by WST-8 assay (left panel) and Calcein assay (right panel). The neuroprotective actions of EF-(S7A)HN or EF-HNG-KKK in the absence of EFLIVIKS in the presence of 25 μM Aβ1-43 [EF-S7A-HN in EFLIVIKS (−) or EF-HNG-KKK in EFLIVIKS (−)] were shown (n=1). In addition to no treatment (no T), Aβ treatment alone, other controls were the negative effects of 100 μM EFLIVIKS on the rescue function of 10 μM EF-HN (EF-HN) or of EF-HNG (EF-HNG) against 25 μM Aβ1-43.

FIG. 16 shows representative cellular Calcein fluorescence microscopic photographs representing blocking effect of EFLIVIKS on the neuroprotective action of EF-(S7A)HN, EF-HNG-KKK, EF-HN or EF-HG.

The representative views of cellular Calcein fluorescence are indicated, when primary neurons were treated with or without 25 μM Aβ1-43 in the presence or absence of indicated combinations of the peptides [EFLIVIKS, 100 μM; EF-HNG-KKK, 10 μM; EF-(S7A)HN (abbreviated as EF-S7A), 10 μM; EF-HN, 10 μM; EF-HNG, 10 μM].

FIG. 17 shows Immunoprecipitation-Western blot assay results indicating positive or negative dimerization of HN or other HN derivatives.

A: The C-terminally FLAG-tagged HN derivative peptide [HN-DYKDDDDK (HNFLAG), (S7A)HN-DYKDDDDK (S7A-FLAG), EFLIVIKS-(S7A)HN-DYKDDDDK (EF-S7A-FLAG)] was precipitated with Ni-beads immobilizing the (His)₆-tagged peptide [His)₆-HN (His-HN), (His)₆-(S7A)HN (His-S7A), or (His)₆-EFLIVIKS-(S7A)HN (His-EF-S7A)]. The precipitate was analyzed with Tris/Tricine gel electrophoresis and anti-FLAG antibody. The numbers in the left side of the panel indicate molecular weights in kDa.

B: The FLAG-tagged HN derivative peptide (His-HN or His-EF-S7A) was precipitated with the (His)₆-tagged HN derivative peptide (HN-FLAG or EF-S7A-FLAG) in the presence of excess amounts of each competitor [none (−), 100 nmol tag-free HN (HN), 100 mmol EFLIVIKS (EF), 100 mmol (S7A)HN(S7A), 10 nmol HNG (EING), or 10 nmol HNG-KKK (HNG-KKK)]. The FLAG peptides in the precipitate were detected by M2 antibody. The bars in the left side of the panels indicate molecular weights in kDa (30, 21.5, 14.3, 6.5, 3.4 from the top to the bottom).

FIG. 18 shows representative cellular Calcein fluorescence microscopic photographs representing results of neurocytotoxicity study in the presence or absence EF-AGA-HNG or EF-HNG at indicated concentrations.

Primary cultured neurons were treated with 25 μM Aβ1-43 and cultured in the presence or absence of indicated concentrations of EF-AGA-HNG or AGA-HNG. Seventy-two hr after the onset of Aβ treatment, alive neurons were stained with Calcein-AM. The representative views of cellular Calcein fluorescence are indicated.

FIG. 19 shows graphs representing results of neurocytotoxicity study in the presence or absence EF-AGA-HNG, EF-HNG, or AGA-HNG.

Effects of EF-AGA-HNG on neuronal cytotoxicity by Aβ or FAD genes. Primary cultured neurons were treated with 25 μM Aβ1-43 and cultured in the presence of increasing concentrations of EF-AGA-HNG, EF-HNG, or AGA-HNG, as indicated. Cell viability was measured 72 hr after Aβ treatment by Calcein fluorescence assay (upper panel) or WST-8 absorbance assay (lower panel). The values are indicated as the percentage of the Calcein fluorescence (upper panel) or the specific WST-8 absorbance (the WST-8 absorbance-non-specific absorbance) obtained from cells without Aβ treatment.

FIG. 20 shows graphs representing results of neurocytotoxicity study in the presence or absence EF-AGA-HNG, EF-HNG, or AGA-HNG.

F11 cells were transfected with either FAD gene (V642I-APP cDNA, NL-APP cDNA, M146L-PS1 cDNA, N141I-PS2 cDNA) and cultured in the presence of increasing concentrations of EF-AGA-HNG (squares and lines), EF-HNG (triangles and lines), or AGA-HNG (circles and bold lines). Cell mortality was measured 72 hr after transfection by Trypan blue exclusion assay.

FIG. 21 shows immunoblot photographs representing results of expression study of V642I-APP, NL-APP, M146L-PS1, or N141I-PS2 in the presence or absence EF-AGA-HNG or EF-HNG at indicated concentrations.

F11 cells were transfected with V642I-APP cDNA, NL-APP cDNA, M146L-PS1 cDNA, or N141I-PS2 cDNA in pcDNA or pcDNA (vec) and cultured in the presence or absence of increasing concentrations of EF-AGA-HNG (1 fM, 10 fM, 100 fM, 1 pM, 10 pM, 100 pM, 1 nM, 10 nM, 100 nM) or AGA-HNG (1 pM, 10 pM, 100 pM, 1 nM). Cell lysates were submitted to SDS-PAGE and immunoblot analysis with anti-APP antibody 22C11 (upper panels; the arrow indicates the holoproteins of V642I-APP (panel A) or NL-APP (panel B)), anti-PS1 antibody [left lower panel; the upper and lower arrows indicate the holoprotein and the N-terminal fragment (NTF) of M146L-PS1, respectively] (panel C), or anti-PS2 antibody [right lower panel; the upper and lower arrows indicate the holoprotein and the C-terminal fragment (CTF) of N141I-PS2, respectively] (panel D). The numbers in the left side of the panels indicate molecular weights in kDa.

### DETAILED DESCRIPTION OF THE INVENTION

There is a need for treating neurodegenerative disorders such as Alzheimer's disease. One way to establish useful therapies for these diseases is to control the occurrence of neuronal cell death [2-18]. The present invention relates to new HN derivative polypeptides that serve as neuroprotectants, and their use to treat neurodegenerative disorders, both after such disorders have been diagnosed, and before the onset of these disorders as preventive agents for patients who are believed to be at risk for developing a neurodegenerative disorder, such as Alzheimer's disease.

The new HN derivatives are based on the amino acid sequence of the wild-type HN, but each of the derivatives has additional and/or substitute amino acids that significantly increase the neuroprotectant activity of the HN derivative compared to the naturally occurring HN polypeptide. The new HN derivatives are described below, as well as methods for making the derivative polypeptides and pharmaceutical compositions and formulations containing the HN derivatives.

Neuroprotectant Humanin Derivatives

The HN derivatives are polypeptides that are similar to wild-type HN, but have various amino acids added or substituted in the HN sequence (SEQ ID NO:1) to create new polypeptides that have significantly improved neuroprotectant activity compared to the original, wild-type HN.

The new HN derivatives are at least about 17 to about 32, e.g., 12, 14, 16, 17, 18, 20, 22, 24, 26, 28, 30, or 32, amino acids in length, and include one or both of two main classes of modifications of the wild-type HN polypeptide.

The first modification is D-racemization of one of the 24 amino acids of wild-type HN. For example, the Ser¹⁴ or Ser⁷ amino acid, or both, can be converted from an L-isomer to a D-isomer.

The second modification promotes multimerization, e.g., dimerization, of the HN derivative compared to the wild-type HN. For example, dimerization can be potentiated by the addition of a multimerization peptide, such as EFLRVIKS or EFLIVKS, to the HN polypeptide

Other modifications are also possible, and can be combined with one or more of the two main modifications. For example, in certain HN derivatives, the Arg⁴ and Phe⁶ amino acids can be substituted with Alanine to form R4A/6A, or AGA-HN for short. In addition, Ser¹⁴ can be substituted by Glycine to form S14G HN, also referred to as HNG for short, or substituted by Proline to form S14P, also referred to as HNP for short. In other embodiments, Ser⁷ is substituted, e.g., by Alanine, to form S7A HN.

Combinations of any two of more of these modifications in a single HN derivative are also possible.

Table 1 lists HN and various representative HN derivatives that each embodies one or more of the modifications that are within the HN derivatives.

TABLE 1
Humanin (HN) Derivatives

| | | |
|---|---|---|
| Peptides | SEQ ID NO: | Sequence |

| | | |
|---|---|---|
| P-S14 HN | 4 | MAPRGFSCLLLLT(p-S)EIDLPVKRRA |
| P-S7 HN | 5 | MAPRGF(p-S)CLLLLTSEIDLPVKRRA |
| P-S7/14 HN | 6 | MAPRGF(p-S)CLLLLT(p-S)EIDLPVKRRA |
| D-Ser14 HN | 7 | MAPRGFSCLLLLT(D-S)EIDLPVKRRA |
| D-Ser7 HN | 8 | MAPRGF(D-Ser)CLLLLTSEIDLPVKRRA |
| D-Ser7/14 HN | 9 | MAPRGF(D-Ser)CLLLLT(D-Ser)EIDLPVKRRA |
| AGA-(D-Ser14) HN | 10 | MAPAGASCLLLLT(D-Ser)EIDLPVKRRA |
| AGA-(D-Ser14) HN17 | 11 | PAGASCLLLLT(D-Ser)EIDLP |
| EF-(S7A)HN | 15 | EFLIVIKSMAPRGFACLLLLTSEIDLPVKRRA |
| EF-HNG-KKK | 16 | EFLIVIKSMAPRGFSCLLLLTGEIDLPVKKKK |
| EF-HN | 17 | EFLIVIKSMAPRGFSCLLLLTSEIDLPVKRRA |
| EH-HNA | 18 | EFLIVIKSMAPRGFSALLLLTSEIDLPVKRRA |
| EF-HNG | 19 | EFLIVIKSMAPRGFSCLLLLTGEIDLPVKRRA |
| EF-AGA-HNG | 22 | EFLIVIKSMAPAGASCLLLLTGEIDLPVKRRA |

Note:
Amino acid residues not present in a naturally occurring HN are in bold face font. “p-S” represents phosphorylated L-Ser.

The present invention shows that the Ser residues at positions 7 and 14 in HN play essential roles in the activity regulation of HN. The specified role that the Ser¹⁴ residue plays is the regulation of the HN activity through D-racemation. When the L-Ser¹⁴ residue was substituted to D-Ser, the cytoprotective action of HN was extremely potentiated. This potency was equivalent to that of HNG, HN with S14G substitution. It is thus most likely that S14G substitution mimics D-racemation of Ser¹⁴ and that authentic HN, which is effective at low FM levels, is a precursor form, which is further activated by Ser¹⁴ racemation to be effective at low nM levels. While there is a possibility that such a conversion is simply a physical process, there is an alternative possibility that this conversion is mediated by L- to D-Ser converting enzymes, Ser racemases. It is worth investigating the presence of an enzyme converting L-Ser¹⁴ of HN to D-Ser and to identify the enzyme, if present, because determining its localization and regulation would contribute significantly to understanding how to activate HN by methods other than exogenous administration of HN peptide. Most recently, Wolosker et al. [34] have identified and cloned the first mammalian Ser racemase that converts free L-Ser to D-Ser in glial cells.

The Ser⁷ residue plays a role different from that of Ser¹⁴. S7A substitution nullified the action of HN, while Ser⁷ racemation had no influence. Loss of HN action by S7A substitution was recovered by the fusion with EFLIVIKS, suggesting that Ser⁷ plays a role similar to that of EFLIVIKS fusion. Given the fact that EFLIVIKS or EFLIVKS is the established amino acid sequence to form a dimer [32, 33] and that EFLIVIKS alone had no cytoprotective action, it is most likely that Ser⁷ is essential for HN to self-dimerize and that self-dimerization of HN is essential for the action of HN. In fact, the precipitation experiments using two kinds of distinctly tagged HN an HN derivatives revealed that HN can self-dimerize and that S7A substitution loses its self-dimerization activity. This experiment also provides evidence that EF-(S7A)HN self-dimerizes through the EFLIVIKS region. The finding that EFLIVIKS fusion allowed (S7A)HN to exert cytoprotection as potent as EFLIVIKS-fused HN indicates that (S7A)HN is not able to exert cytoprotection simply due to its inability to self-dimerize. On the contrary, it is less likely that EFLWVIKS fusion-induced potentiation of the actions of (S7A)HN and HN was attributable to increased stability of these peptides through or not through their dimerization, because the apparent stability of HN, HNG, or (S7A)HN in the culture medium was little or only marginally affected by their fusion with EFLIVIKS [by 72-hr incubation of 10 μM peptides in the cultured medium, the amount of HN, HNG, or (S7A)HN became about ⅔ of the amount at time zero, whereas the amount of EF-HN, EF-HNG, or EF-(S7A)HN was virtually unaltered (data not shown)]. This level of stability alterations could not account for the drastic potentiation of the rescue action by the fusion with EFLIVIKS.

There are other multiple lines of evidence supporting that EFLIVIKS fusion functions as a dimerization tag. Firstly, a similar observation was obtained from HNG-KKK: HNG-KKK had no action, whereas EF-HNG-KKK exerted potent cytoprotection. In this case, EF-HNG-KKK was nearly 10 times less potent than EF-HNG. It is therefore conceivable that the C-terminal KKK substitution attenuated dimerization activity of HNG, that the N-terminal EFLIVIKS fusion antagonized this negative effect, and that the balance between the stimulatory effect on dimerization by N-terminal EFLIVIKS and the inhibitory effect by C-terminal KKKK resulted in the lower potency of the action of EF-FING-KKK than that of HNG. Secondly, isolated EFLIVIKS peptide selectively blocked the neuroprotective functions of EF-(S7A)HN and EF-HNG-KK, but not that of EF-HN or EF-HNG, providing strong evidence that neuroprotection by EF-(S7A)HN and EF-HNG-KKK is through dimerization of fused EFLIVIKS. Thirdly, an excess amount of HING-KKK was not able to displace HN-HN interaction, when the same concentration of HNG could do so. This provides definite evidence that HNG-KKK has little activity to form a complex with HN. Finally, EFLIVIKS fusion had no effect on (C8A)HN. This indicates that the functional potentiation by EFLIVIKS fusion is not due to physical effects on the fused HN peptides.

The observation that EFLIVIKS fusion failed to potentiate the action of (C8A)HN suggests that there are two different kinds of null mutants in HN: one is (S7A)HN or HNG-KKK, whose action is recovered by EFLIVIKS fusion, and another is (C8A)HN, whose action is not recovered by this procedure. Considering the fact that (C8A)HN is shown to be a receptor-nonbinding mutant [14], it is thus most likely that there are at least two different steps for HN to exert cytoprotective action: one is dimerization (or oligomerization) and the other is a post-dimerization process involving the binding of HN to a putative receptor. It is therefore conceivable that the putative HN receptor may undergo dimerization or oligomerization in response to HN binding. Since the action of HN is mediated by a certain tyrosine kinase system [14], this notion is consistent with the well-established fact that tyrosine-kinase-triggering growth factors and cytokines exert their actions via dimerization, which is essential for receptor dimerization or oligomerization.

Thus, the present invention has uncovered different, essential roles of two Ser residues, each contributing to the potentiation of the rescue function of HN in unique ways. One is Ser¹⁴ racemation and another is dimerization in a Ser⁷-involving manner. While these findings provide a novel insight into the action mechanism of HN, as discussed above, they were also useful in generating more potent HN derivatives. For example, (R4A/F6A)HNG, abbreviated AGA-HNG here, exerts highly potent cytoprotection: half protection at 10-30 pM and full protection at 100-300 pM. Considering the specified role of Ser¹⁴, the present inventors have examined the effect of (R4A/F6A)HN with D-Ser¹⁴ and found that this peptide exerts cytoprotection with potency and efficacy very similar to those of (R4A/F6A)HN with S14G (AGA-HNG), again consistent with the notion that S14G substitution mimics Ser¹⁴ racemation. The inventors have also found that EFLIVIKS-fused AGA-HNG is even more potent than AGA-HNG, again suggesting that EFLIVIKS fusion and S14G replacement potentiate the action of HN in different mechanisms. EFLIVIKS-fused AGA-HNG, which is fully cytoprotective at 10 pM, a concentration 1/2500000 of the employed neurotoxic Aβ concentration, is the most potent HN derivative that has been identified. These peptides will contribute significantly to the understanding of how HN protects neuronal cells from AD-related insults as well as to the development of clinically useful HN derivatives.

The invention includes HN derivatives that are polypeptides that have a sequence that is encoded by, or is substantially identical to the polypeptides encoded by, the nucleic acids of the invention (e.g., polypeptides that are substantially identical to a polypeptide encoded either SEQ ID NOs: 4, 5, 6, 7, 8, 9, 10, 11, 15, 16, 17, 18, 19, or 22). The nucleic acids may be a DNA or an RNA. The nucleic acid sequences can be deduced from the amino acid sequence according to the genetic code, for example, of mammals, other eukaryotes, or prokaryotes. A nucleic acid sequence encoding the HN polypeptide may be 5′-atg gct cca cga ggg ttc agc tgt ctc tta ctt tta acc agt gaa att gac ctg ccc gtg aag agg cgg gca-3′ (SEQ ID NO: 26).

The terms “protein” and “polypeptide” are used herein interchangeably to describe any chain of amino acids, regardless of length or post-translational modification (for example, glycosylation or phosphorylation). Thus, the terms “HN polypeptides” or “HN derivatives” include recombinantly or synthetically produced HN derivative polypeptides that include one or more of the modifications of wild-type HN as described herein. The term also encompasses polypeptides that have an added amino-terminal methionine (useful for expression in prokaryotic cells).

The new HN derivatives described herein can be encoded by any of the nucleic acid molecules, and include biologically active mutants, truncated forms, and fusion polypeptides that have at least the same, and preferably greater, neuroprotective activity than wild-type HN. These polypeptides can be prepared for a variety of uses, including, but not limited to, the generation of antibodies, for the identification of other cellular gene products or compounds that can modulate the activity or expression of nucleic acids or HN derivatives of the invention, and as pharmaceutical reagents useful for the treatment, inhibition, or prevention of cytotoxicity associated with neurodegenerative disorders.

In some embodiments, non-HN derivatives, such as multimerization peptides, can be fused to the N-terminus or C-terminus of the HN derivative. The resulting fusion polypeptide is also an HN derivative, and can include a moiety that has a high affinity for a ligand. For example, the fusion protein can be a GST-HN derivative in which the HN derivative sequences are fused to the C-terminus of the GST sequences. Such fusion proteins can facilitate the purification of recombinant HN derivatives. Alternatively, the fusion protein can be an HN derivative containing a heterologous signal sequence at its N-terminus. In certain host cells (e.g., mammalian host cells), secretion of the HN derivatives can be increased through use of a heterologous signal sequence.

Fusion proteins can also include all or a part of a serum protein, e.g., an IgG constant region, or human serum albumin. The HN derivative fusion polypeptides of the invention can be incorporated into pharmaceutical compositions and administered to a subject in vivo. The HN derivative fusion proteins can be used to affect the bioavailability of an HN derivative substrate.

Expression vectors are commercially available that already encode a fusion moiety (e.g., a multimerization peptide such as EFLIVIKS, or a GST polypeptide). An HN derivative-encoding nucleic acid can be cloned into such an expression vector such that the fusion moiety is linked in-frame to the HN derivative.

HN derivatives can be substantially pure polypeptides, including those that include an intact signal sequence, and secreted forms of the derivatives. Especially preferred are HN derivatives that are soluble under normal physiological conditions.

The invention also encompasses FN derivatives that are functionally equivalent to the specific HN derivatives named herein. The HN derivatives of the invention are equivalent to the specific named FN derivatives in that they possess the same neuroprotectant activity in a biological system, such as in an animal, as the HN derivatives specifically named herein. These additional HN derivatives have at least 60%, and can have 70%, 75%, 80%, 85%, 90%, or even 95% or more of the HN derivatives of the invention (Table 1), and have at least the same neuroprotectant activity of wild-type HN. In the case of an polynucleotide which is longer than or equivalent in length to the reference sequence, e.g., HN (SEQ ID NO: 1), the comparison is made with the full length of the reference sequence. Where the polynucleotide is shorter than the reference sequence, e.g., shorter than SEQ ID NO: 1, the comparison is made to segment of the reference sequence of the same length. Comparisons of the neuroprotectant activity are generally based on an assay of biological activity in which equal concentrations of the polypeptides are used and compared.

Functionally equivalent HN derivatives can be those, for example, that contain additional or substituted amino acid residues, as long as they include one or more modifications from the two main classes of modifications described herein. Substitutions may be made on the basis of similarity in polarity, charge, solubility, hydrophobicity, hydrophilicity, and/or the amphipathic nature of the residues involved. For example, a is functionally equivalent HN derivative is one in which one or two non-essential amino acids is replaced or removed, e.g., replaced by a conservative amino acid substitution. A “conservative amino acid substitution” is one in which an amino acid residue is replaced with an amino acid residue having a similar side chain. Families of amino acid residues having similar side chains have been defined in the art. These families include amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), nonpolar side chains (e.g., alanine, valine, leucine, isoleucine, proline, phenylalanine, methionine, tryptophan), beta-branched side chains (e.g., threonine, valine, isoleucine) and aromatic side chains (e.g., tyrosine, phenylalanine, tryptophan, histidine). Thus, a predicted nonessential amino acid residue in an HN polypeptide can be replaced with another amino acid residue from the same side chain family.

Although there are no particular limitations on the number of amino acid residues to be mutated, it is considered that the number of the residues to be mutated by substitution, deletion, and/or insertion in a amino acid sequence, is generally 15 residues or less, preferably 12 residues or less, more preferably 10 residues or less, and even more preferably 8 residues or less (for example, 5 residues or less). There is no limitation on the number of the amino acids to be added, so long as the neuroprotectant activity is maintained. Artificially produced amino acid sequences and naturally occurring polypeptide sequences are included in the amino acid sequence wherein the amino acids have been substituted, deleted, inserted, and/or added.

Ala-scanning experiment of HNG17 (³PRGFSCLLLLTGEIDLP¹⁹) has revealed that substitution of Ala for either Pro³, Cys⁸, Leu⁹, Leu¹², Thr¹³, Ser¹⁴, or Pro¹⁹ results in non-protective peptides at 10 nM against four FAD genes as well as Aβ1-43, whereas Ala substitution for any other residues, including Ser⁷, do not affect the cytoprotective actions of HNG17 at 10 nM (WO01/21787). This finding elucidates that the 7 amino acids in HN derivatives (corresponding amino acids of Pro³, Cys⁸, Leu⁹, Leu¹², Thr¹³, Ser¹⁴, and Pro¹⁹ of HN17) are critical for neuroprotection. Therefore, polypeptides having different amino acid sequences can be prepared by modification, such as substitution, deletion, and/or insertion, of residues other than the essential amino acids.

What is much more important, is that even the 7 amino acids, mentioned above as essential amino acids for the activity, may be also substituted with other amino acids. For example, the polypeptide retains the neuroprotective activity even when the Gly at position 12 of HNG-17 is Ser (that is HN-17). In addition, a synthetic polypeptide wherein the position corresponding to the Leu at position 7 is substituted with Arg, demonstrated a neuroprotective activity similar to that of HN (WO01/21787). Furthermore, an HNG derivative with basic amino acid, such as His, Arg, or Lys, for the Cys corresponding to position 6 of the HNG-17 shows neuroprotective activity. According to these facts, preparation of polypeptides with equivalent or higher neuronal death suppression activity to the polypeptides are expected by mutating amino acids that are non-essential and/or essential for the activity of these polypeptides.

An HN derivative of the present invention includes polypeptides that protects a neuronal cell from cytotoxicity and having an amino acid sequence consisting of Formula (I):

(I)
Pro—Xn₁—(Cys/bXaa)—(Leu/Arg)—Xn₂—Leu—Thr—
(Gly/Ser)*—Xn₃—Pro

Herein, “Cys/bXaa” indicates Cys or a basic amino acid; “(Leu/Arg)” indicates Leu or Arg; “(Gly/Ser)” indicates Gly or Ser; and Xn₁, Xn₂, and Xn₃ independently indicate arbitrary amino acids not more than 10 residues, respectively. The position of the amino acid shown by an asterisk in Formula (I) corresponds to the position of ¹⁴Ser of Humanin. An HN derivative of the invention may include at least one D-amino acid or one phosphorylated amino acid in the amino acid sequence of Formula (I), preferably at the position shown by the asterisk. Alternatively, an HN derivative of the invention includes one or more amino acids having an activity of forming a multimer, in addition to the amino acid sequence of Formula (I). A polypeptide that has the amino acid sequence as above may be also expressed as:

(II)
Pro—(Xaa)1-10—(Cys/bXaa)—(Leu/Arg)—(Xaa)1-10—Leu—
Thr—(Gly/Ser)*—(Xaa)1-10—Pro

(wherein Xaa indicates an arbitrary amino acid; “(Xaa)ₘ₋ₙ” indicates m to n residues of arbitrary amino acids; “bXaa” indicates a basic amino acid; “Cys/bXaa” indicates Cys or a basic amino acid; “(Leu/Arg)” indicates Leu or Arg; and “(Gly/Ser)” indicates Gly or Ser).

Basic amino acids refer to amino acids in which its R group (side chain) is positively charged at pH7.0. Examples of natural basic amino acids include Arg, Lys, and His. The amino acid sequences of a polypeptide of this invention that has Arg, Lys, or His as the basic amino acids can be represented, for example, as:
Pro-Xn₁-(Cys/Arg/Lys/His)-(Leu/Arg)-Xn₂-Leu-Thr-(Gly/Ser)*-Xn₃-Pro (III)
(wherein “(Cys/Arg/Lys/His)” indicates Cys, Arg, Lys, or His; “(Leu/Arg)” indicates Leu or Arg; “(Gly/Ser)” indicates Gly or Ser; and Xn₁, Xn₂, and Xn₃ independently indicate arbitrary amino acids not more than 10 residues, respectively). Herein, Arg and Lys are particularly preferable as the basic amino acid at this position. However, when the polypeptide has an additional amino acids that form multimer, such as EFLIVIKS (SEQ ID NO: 20) or EFLIVKS (SEQ ID NO: 24), the amino acid at the position of “Cys/bXaa” or “(Cys/Arg/Lys/His)” may not be limited to Cys or a basic amino acid but may be any amino acids.

Preferably, Xn₁, Xn₂, and Xn₃ are independently arbitrary amino acids of 2 to 6, 0 to 4, and 2 to 6 residues, respectively (that is, Xn₁=(Xaa)₂₋₆, Xn₂=(Xaa)₀₋₄, and Xn₃=(Xaa)₂₋₆); more preferably 3 to 5, 1 to 3, and 3 to 5 residues, respectively (that is, Xn₁=(Xaa)₃₋₅, Xn₂=(aa)₁₋₃, and Xn₃=(Xaa)₃₋₅); and most preferably 4, 2, and 4 residues, respectively (that is, Xn₁=(Xaa)₄, Xn₂=(Xaa)₂, and Xn₃=(Xaa)₄). Added amino acids of about 6 residues sometimes forms an alpha-helix and behaves like a single amino acid residue. Accordingly, a derivative polypeptide of the present invention may be a polypeptide wherein arbitrary amino acids with no more than 6 residues are added to all or any one of Xn₁, Xn₂, and Xn₃ consisting of arbitrary amino acids of 4 residues, 2 residues, and 4 residues, respectively.

Such polypeptide may be prepared according to known peptide synthesis techniques, and also by the expression of a DNA that encodes such polypeptides.

Preferably, the sequence of Xn₁ includes, for example, sequences consisting of (Arg/Ala)-(Gly/Ala)-(Phe/Ala)-(Ser/Ala), and sequences with conservative substitution thereof. Herein, for example, “Arg/Ala” indicates Arg or Ala (“/” indicates that it is either one of the residues; the same is indicated throughout the description herein). Examples of such sequences include Arg-Gly-Phe-Ser, Ala-Gly-Phe-Ser, Arg-Ala-Phe-Ser, Arg-Gly-Ala-Ser, Arg-Gly-Phe-Ala, and so on. Other examples include Arg-Gly-Ala-Ala, Arg-Ala-Phe-Ala, Arg-Ala-Ala-Ser, Arg-Ala-Ala-Ala, Ala-Gly-Phe-Ala, Ala-Gly-Ala-Ser, Ala-Gly-Ala-Ala, Ala-Ala-Phe-Ser, Ala-Ala-Phe-Ala, Ala-Ala-Ala-Ser, Ala-Ala-Ala-Ala, and such. Conservative substitution can be exemplified by substitution within a group of amino acids, corresponding to conservative substitution, which is described above. On the other hand, the sequence of Xn₂ preferably includes, for example, sequences consisting of (Leu/Ala)-(Leu/Ala), and sequences with conservative substitution thereof. Such sequences include Leu-Leu, Ala-Leu, Leu-Ala, and such. Ala-Ala can be also exemplified as such sequences. Furthermore, the sequence of Xn₃ preferably includes, for example, sequences consisting of (Glu/Ala)-(Ile/Ala)-(Asp/Ala)-(Leu/Ala), and sequences with conservative substitution thereof. Such examples include Glu-Ile-Asp-Leu, Ala-Ble-Asp-Leu, Glu-Ala-Asp-Leu, Glu-Ile-Ala-Leu, Glu-Ile-Asp-Ala, and so on. Other examples are Glu-Ile-Ala-Ala, Glu-Ala-Asp-Ala, Glu-Ala-Ala-Leu, Glu-Ala-Ala-Ala, Ala-Ile-Asp-Ala, Ala-Ile-Ala-Leu, Ala-Ile-Ala-Ala, Ala-Ala-Asp-Leu, Ala-Ala-Asp-Ala, Ala-Ala-Ala-Leu, Ala-Ala-Ala-Ala, and so on. The sequences of Xn₁, Xn₂, and Xn₃ may be selected from arbitrary combinations.

Cytotoxicity of neuronal cells is induced by the expression of APP, PS-1, or PS-2 is mutants (for example, V642I/F/G APP, NL-APP, M146L PS-1, and N141I PS-2) in established neuronal cell lines (for example, F11 cells) and primary neuronal cultures (for example, rat brain cortical primary culture); and also by the addition of Aβ (for example, Aβ1-43) to primary neuronal cultures. The term “protection of a neuronal cell from cytotoxicity” herein defines to suppress at least one of the cytotoxicities of neuronal cells including those mentioned above. Specifically, the HN derivatines of the present invention include those, that suppress at least any one of these neuronal deaths caused by an APP, PS-1, or PS-2 mutant or Aβ. The suppression of cell death doesn't have to be a complete suppression so long as the suppression is significant. The activity of proteins to suppress neuronal death can be detected according to the method described in the Examples, or by other published methods (see for example, International Publication No. WO00/14204).

More specifically, a method as follows can be exemplified: (1) transfect neurons (for example, F11 cells) with vectors expressing FAD genes, such as V642I/F/G APP, NL-APP, M146L PS-1, and N141I PS-2, alone or in combination with a vector expressing a polypeptide to be examined; (2) cultivating the cells for a defined period (for example, 72 hours); and (3) detecting level of cell death by trypan blue exclusion assay. Alternatively, a polypeptide to be examined is prepared in advance, and cell death may be measured upon transfection of FAD genes into cells in the presence or absence of the polypeptide. FAD genes may be also conditionally expressed using an inductive promoter. A polypeptide is determined to suppress neuronal death, when the cell death under the existence of the polypeptide is significantly decreased in comparison to those induced in the absence of the polypeptide to be examined. Additionally, other cells such as primary cultured neurons may be used, and induction of cell death can be also carried out by the addition of Aβ. Cell death can be measured by detecting morphological changes, LDH release, or apoptosis (morphological changes of the nucleus, fragmentation of DNA, and such) in addition to trypan blue exclusion.

Polypeptides that are functionally equivalent to the HN derivatives of the invention can be made using random mutagenesis on the encoding nucleic acids by techniques well known to those skilled in the art. It is more likely, however, that such polypeptides will be generated by site-directed mutagenesis (again using techniques well known to those skilled in the art). These polypeptides may have increased functionality or decreased functionality, but must have at least the neuroprotective effect of HN.

Mutations within the coding sequence of nucleic acid molecules of the invention can be made to generate variant sequences that are better suited for expression in a selected host cell. For example, N-linked glycosylation sites can be altered or eliminated to achieve, for example, expression of a homogeneous product that is more easily recovered and purified from yeast hosts that are known to hyperglycosylate N-linked sites. To this end, a variety of amino acid substitutions at one or both of the first or third amino acid positions of any one or more of the glycosylation recognition sequences which occur, and/or an amino acid deletion at the second position of any one or more of such recognition sequences, will prevent glycosylation at the modified tripeptide sequence (see, for example, Miyajima et al., EMBO J., 5:1193, 1986).

In many embodiments, the new HN derivatives have a biological neuroprotective activity that is two to three orders of magnitude greater than wild-type HN. In one embodiment, HN derivatives exert neuroprotective functions at concentrations as low as 1-10 pM.

Methods of Making the New HN Derivatives

The polypeptides of the invention can be chemically synthesized (for example, see Creighton, “Proteins: Structures and Molecular Principles,” W.H. Freeman & Co., NY, 1983), or, alternatively, produced by recombinant DNA technology as described herein. For additional guidance, skilled artisans may consult Ausubel et al. (“Current Protocols in Molecular Biology,” 4th edition, John Wiley and Sons, 1995), Sambrook et al. (“Molecular Cloning, A Laboratory Manual,” Cold Spring Harbor Press, Cold Spring Harbor, N.Y., 1989), and, particularly for examples of chemical synthesis Gait, M. J. Ed. (“Oligonucleotide Synthesis,” IRL Press, Oxford, 1984). The method for peptide synthesis may be either solid-phase synthesis or liquid-phase synthesiss (Japanese Biochemical Society edition, “Shin Seikagaku Jikken KozaTanpakushitu (New Course on Biochemistry Experiments, Proteins) VI,” pp. 3-74, Tokyo Kagakudojin, 1992).

The polypeptides of this invention include salts thereof. Such salts are derived from acids or bases of the polypeptides. Specifically, such salts can be exemplified by salts formed with inorganic acids (for example, hydrochloride, phosphate, hydrobromide, hydrosulfate, nitrate, etc.); salts formed with organic acids (for example, acetate, lactate, formate, butyrate, glycolate, propionate, fumarate, maleate, succinate, tartrate, citrate, malate, oxalate, benzoate, methane sulfonate, benzene sulfonate, etc.); and salts formed with bases (for example, ammonium salt, alkali metal salts such as sodium salt and potassium salt, alkaline earth metal salts such as calcium salt and magnesium salt, and salts formed with organic bases, and salts formed with amino acids such as arginine and lysine).

The polypeptide of the present invention may be an peptide-derivative. Herein, the term “an peptide-derivative” refers to molecules that have a form, which has been altered by modification, addition, mutation, substitution, or deletion of functional groups of the polypeptide according to conventional methods. Such alterations of functional groups are carried out, for example, to protect functional groups of the polypeptides, to regulate the stability or histological localization of the polypeptides, or to regulate the activity of the polypeptides, and so on. The polypeptides of the present invention are exemplified by those polypeptides wherein any one of the N-terminus, C-terminus, and functional groups of the polypeptides constituting amino acid side chains are modified by substituents, such as protecting groups. The substituents include, for example, various alkyl groups, acyl groups, amide groups, phosphate groups, amino groups, carboxyl groups, and ester groups; however, the present invention is not limited to these examples.

Further, the polypeptides may be bound to a carrier. For example, the polypeptides of this invention may be bound to polyethylene glycol (PEG), dextran, other polymers, and so on. The polypeptides may have natural and/or unnatural amino acids. Unnatural amino acids are exemplified by homoserine, β-hydroxyvaline, 0-4-hydroxyphenyl tyrosine, a-t-butyl glycine, 2-amino butyrate, a-cyclohexyl glycine, a-phenyl glycine, and such. Further, the peptide bonds of the polypeptides may be appropriately substituted with covalent bonds other than peptide bonds. The sensitivity to peptidases of the polypeptides can be lowered by the substitution to non-peptide bonds, which enhances drug efficacy duration and which offers a wide selection of administration routes. The non-peptide bonds are exemplified by imino bonds, ester bonds, hydrazine bonds, semicarbazide bonds, and azo bonds, but the present invention is not limited to these examples.

In other embodiments, the new HN derivatives can be prepared using standard recombinant technology using known host cells. Any desired mutations can be introduced into Humanin cDNA by the production of synthetic DNA or by site directed mutagenesis. There are no limitations on the number and position of the amino acids to be modified so long as the obtained polypeptide has the neuroprotectant activity. Vectors, preferably expression vectors, containing a nucleic acid encoding an HN derivative are useful for expressing the HN derivatives in vitro and in vivo. The recombinant expression vectors can be designed for expression of HN derivatives in prokaryotic or eukaryotic cells, e.g., E. coli, insect cells (e.g., using baculovirus expression vectors), yeast cells, or mammalian cells. Suitable host cells are discussed further in Goeddel, Gene Expression Technology: Methods in Enzymology 185, Academic Press, San Diego, Calif. (1990). Alternatively, the recombinant expression vector can be transcribed and translated in vitro, for example using T7 promoter regulatory sequences and T7 polymerase.

Expression of proteins in prokaryotes is most often carried out in E. coli with vectors containing constitutive or inducible promoters directing the expression of either fusion or non-fusion proteins. Fusion vectors add a number of amino acids to a protein encoded therein, usually to the amino terminus of the recombinant protein. Such fusion vectors typically serve three purposes: 1) to increase expression of recombinant protein; 2) to increase the solubility of the recombinant protein; and 3) to aid in the purification of the recombinant protein by acting as a ligand in affinity purification. Often, a proteolytic cleavage site is introduced at the junction of the fusion moiety and the recombinant protein to enable separation of the recombinant protein from the fusion moiety subsequent to purification of the fusion protein. Such enzymes, and their cognate recognition sequences, include Factor Xa, thrombin and enterokinase. Typical fusion expression vectors include pGEX (Pharmacia Biotech Inc; Smith, D. B. and Johnson, K. S., 1988, Gene 67:31-40), pMAL (New England Biolabs, Beverly, Mass.) and pRIT5 (Pharmacia, Piscataway, N.J.) which fuse glutathione S-transferase (GST), maltose E binding protein, or protein A, respectively, to the target recombinant protein. Other host-vector systems such as the baculovirus-Sf cell line (Okamoto et al., J. Biol. Chem. 270: 4205-4208, 1995), the pcDNA-CHO cell line (Takahashi et al., J. Biol. Chem. 270: 19041-19045, 1995), or the CMV promoter plasmid-COS cell line (Yamatsuji et al., EMBO J. 15: 498-509, 1996) may be used, but are not limited thereto.

Purified fusion proteins can be used in HN derivative activity assays, (e.g., assays of neuroprotective activity detail herein), or to generate antibodies specific for HN derivatives. To maximize recombinant protein expression in E. coli, the protein is expressed in a host bacterial strain with an impaired capacity to proteolytically cleave the recombinant protein (Gottesman, Gene Expression Technology: Methods in Enzymology 185, Academic Press, San Diego, Calif., 1990, pp. 119-128). Another strategy is to alter the nucleic acid sequence of the nucleic acid to be inserted into an expression vector so that the individual codons for each amino acid are those preferentially utilized in E. coli (Wada et al., 1992, Nucleic Acids Res. 20:2111-2118). Such alteration of nucleic acid sequences of the invention can be carried out by standard DNA synthesis techniques.

The HN derivative expression vector can be a yeast expression vector, a vector for expression in insect cells, e.g., a baculovirus expression vector, or a vector suitable for expression in bacterial, fungal, or mammalian cells. When used in mammalian cells, the expression vector's control functions are often provided by viral regulatory elements. For example, commonly used viral promoters are derived from polyoma, Adenovirus 2, cytomegalovirus, and Simian Virus 40 (SV40).

Recombinant mammalian expression vector can be used to direct expression of the nucleic acid preferentially in a particular cell type (e.g., tissue-specific regulatory elements are used to express the nucleic acid). Non-limiting examples of suitable tissue-specific promoters include the albumin promoter (liver-specific; Pinkert et al. (1987, Genes Dev. 1:268-277), lymphoid-specific promoters (Calame and Eaton, 1988, Adv. Immunol. 43:235-275) in particular promoters of T cell receptors (Winoto and Baltimore, 1989, EMBO J. 8:729-733) and immunoglobulins (Baneiji et al., 1983, Cell 33:729-740; Queen and Baltimore, 1983, Cell 33:741-748), neuron-specific promoters (e.g., the neurofilament promoter; Byrne and Ruddle (1989) Proc. Natl. Acad. Sci. USA 86:5473-5477), pancreas-specific promoters (Edlund et al., 1985, Science 230:912-916), and mammary gland-specific promoters (e.g., milk whey promoter; U.S. Pat. No. 4,873,316 and European Application Publication No. 264,166). Developmentally-regulated promoters are also encompassed, for example, the murine hox promoters (Kessel and Gruss, 1990, Science 249:374-379) and the a-fetoprotein promoter (Campes and Tilghman, 1989, Genes Dev. 3:537-546).

Under some circumstances it is desirable to produce a host cell which includes a nucleic acid encoding all or part of an HN derivative within a recombinant expression vector or an HN derivative nucleic acid molecule containing sequences which allow it to homologously recombine into a specific site of the host cell's genome. A host cell can be any prokaryotic or eukaryotic cell. For example, an HN derivative can be expressed in bacterial cells such as E. coli, insect cells, yeast, or mammalian cells (such as Chinese hamster ovary cells (CHO)) or COS cells. Other suitable host cells are known to those skilled in the art.

Vector DNA can be introduced into host cells via conventional transformation or transfection techniques, e.g., any art-recognized technique for introducing foreign nucleic acid (e.g., DNA) into a host cell, including calcium phosphate or calcium chloride co-precipitation, DEAE-dextran-mediated transfection, lipofection, or electroporation.

The host cells of the invention can be used to produce (i.e., express) an HN derivative, e.g., by culturing a host cell (into which a recombinant expression vector encoding an HN derivative has been introduced) in a suitable medium such that an HN derivative is produced and, optionally isolating an HN derivative from the medium or the host cell.

Alternatively, the polypeptides of the invention can be expressed fused to another polypeptide, for example, a marker polypeptide or fusion partner, such as a multimerization, e.g., dimerization, peptide. A fusion polypeptide is a polypeptide in which at least two polypeptides that are not bound in nature are joined, and can be produced by peptide synthesis, or by expressing nucleic acids wherein the polypeptide encoding regions are ligated in frame. For example, the polypeptide can be fused to a hexa-histidine tag to facilitate purification of bacterially expressed protein or a hemagglutinin tag to facilitate purification of protein expressed in eukaryotic cells. Examples of other polypeptides that is fused to the protein of this invention include arbitrary polypeptides comprising short peptides with few residues, such as tags, and long polypeptides, such as proteins. Specifically, such examples include His tag, HA tag, GFP, maltose binding protein, and glutathione S-transferase (GST). Additionally, antibody fragments (Fc fragment), and such may be also used. Other examples include leader sequence, secretion signal, and preprotein or proprotein sequences, but the present invention is not limited to these examples. Further, a group of polypeptides, that facilitates the polypeptide of this invention to effectively pass the blood-brain barrier, can be fused to the protein of the present invention.

A fusion protein may be readily purified by utilizing an antibody specific for the fusion protein being expressed. For example, a system described by Janknecht et al. allows for the ready purification of non-denatured fusion proteins expressed in human cell lines (Proc. Natl. Acad. Sci., USA, 88:8972-8976, 1991). In this system, the gene of interest is subcloned into a vaccinia recombination plasmid such that the gene's open reading frame is translationally fused to an amino-terminal tag consisting of six histidine residues. Extracts from cells infected with recombinant vaccinia virus are loaded onto Ni²⁺ nitriloacetic acid-agarose columns and histidine-tagged proteins are selectively eluted with imidazole-containing buffers.

Although there is no limitation regarding the number of amino acid residues in the polypeptides of HN derivateves, however, for example, when the polypeptide is used as a pharmaceutical composition, polypeptides of smaller molecular size are generally preferred. Absence of portions (for example, amino acid residues or functional groups) unnecessary for the activity decreases antigenicity, and non-specific interactions with other molecules can be avoided which as a result is expected to reduce unfavorable side effects. The polypeptides of the present invention consist of preferably 500 amino acid residues or less, more preferably 100 residues or less, much more preferably 50 residues or less, and even more preferably 30 residues or less. The average molecular weight of the polypeptides is preferably 60 kDa or less, more preferably 15 kDa or less, more preferably 6 kDa or less, and even more preferably 4 kDa or less.

Formulations and Uses of the HN Derivatives

Pharmaceutical compositions for use in accordance with the present invention can be formulated in a conventional manner using one or more physiologically acceptable carriers or excipients. A vector expressing an HN derivative as a medicament may be used to perform gene therapy. Secreting types of the polypeptides, or polypeptides, modified with secretion signal attachment, may be expressed for the gene therapy. Administration methods for the vectors include in vivo and ex vivo methods. Vector systems for gene therapy include: adenovirus vector; adenovirus-associated virus (AAV) vector; herpesvirus vector (all refer to Robbins and Ghivizzani, Pharmacol. Ther. 80: 3547, 1998); retrovirus vector (Engel and Kohn, Front. Biosci. 4: e26-33, 1999); lentivirus vector (Lundstrom, K., 1999, J. Recept. Signal. Transduct. Res. 19: 673-686); and such, but are not limited thereto.

Neuroprotective compositions and their physiologically acceptable salts and solvates can be formulated for administration by various methods. For example, administration can be parenteral, intravenous, subcutaneous, intramuscular, intracranial, intraorbital, ophthalmic, intraventricular, intracapsular, intraspinal, intracisternal, intraperitoneal, transmucosal, or oral. Suitable administration methods also include percutaneous, intranasal, transbronchial, intraspinal, intradural or intracerebroventricular administrations. The compounds can be formulated in various ways, according to the route of administration.

For example, to protect cerebral neurons against degeneration in Alzheimer's disease treatment, it is preferable to administer a polypeptide of the present invention so that the concentration around the target cells is sufficient to effectively suppress neurodegeneration. Specifically, HN derivatives that have equivalent protective action against neuronal death with HN(P-S 14 HN, P-S7 HN, P-S14/7 HN, D-Ser7 HN), should be administered at a concentration of at least 1 nM or more, preferably 10, 100, or 500 nM or more, and more preferably 1, 5, 10, or 30 μM or more. HN derivatives that have equivalent protective action against neuronal death with EF-HN (or EF-(S7A)UN), should be administered at a concentration of 10, or 100 pM or more, preferably 1, 10, 50, 100 or 300 nM or more. HN derivatives that have equivalent protective action against neuronal death with D-Ser14HN (D-Ser7/14HN, S7A HNG17, EF-HNG-KKK), should be administered at a concentration of 1, 5, 10, 50, or 100 pM or more, preferably 1, 10, or 30 nM or more. HN derivatives that have equivalent protective action against neuronal death with AGA-D-Ser14HN (AGA-D-Ser14HN17, EF-HNG, AGA-HNG), should be administered at a concentration of at least 100 fM or more, preferably, 1, 5, 10, 50, or 100 pM or more, preferably 1 nM or more. HN derivatives that have equivalent protective action against neuronal death with EF-AGA-HNG, should be administered at a concentration of at least 1, 5, 10, 50, or 100 fM or more, preferably, 1, 5, 10, or 30 pM or more. The dosage to achieve these concentrations can be appropriately determined taking the administration route into consideration.

For oral administration, the pharmaceutical compositions can take the form of, for example, tablets or capsules prepared by conventional means with pharmaceutically acceptable excipients such as binding agents (for example, pregelatinised maize starch, polyvinylpyrrolidone or hydroxypropyl methylcellulose); fillers (for example, lactose, microcrystalline cellulose or calcium hydrogen phosphate); lubricants (for example, magnesium stearate, talc or silica); disintegrants (for example, potato starch or sodium starch glycolate); or wetting agents (for example, sodium lauryl sulphate). The tablets can be coated by methods well known in the art.

Liquid preparations for oral administration can take the form of, for example, solutions, syrups or suspensions, or they can be presented as a dry product for constitution with water or other suitable vehicle before use. Such liquid preparations can be prepared by conventional means with pharmaceutically acceptable additives such as suspending agents (for example, sorbitol syrup, cellulose derivatives or hydrogenated edible fats); emulsifying agents (for example, lecithin may serve this function, or acacia); non-aqueous vehicles (for example, almond oil, oily esters, ethyl alcohol or fractionated vegetable oils); and preservatives (for example, methyl or propyl-p-hydroxybenzoates or sorbic acid). The preparations can also contain buffer salts, flavoring, coloring and sweetening agents as appropriate. Preparations for oral administration can be suitably formulated to give controlled release of the active compound.

For buccal or sublingual administration the compositions can take the form of tablets or lozenges formulated in conventional manner.

The compounds can be formulated for parenteral administration by injection, for example, by bolus injection or continuous infusion. Formulations for injection can be presented in unit dosage form, for example, in ampoules or in multi-dose containers, with an added preservative. The compositions can take such forms as suspensions, solutions or emulsions in oily or aqueous vehicles, and can contain formulatory agents such as suspending, stabilizing and/or dispersing agents. Alternatively, the active ingredient can be in powder form for constitution with a suitable vehicle, for example, sterile pyrogen-free water, before use.

The compounds can also be formulated in rectal compositions such as suppositories or retention enemas, for example, containing conventional suppository bases such as cocoa butter or other glycerides.

In addition to the formulations described previously, the compounds can also be formulated as a depot preparation. Such long acting formulations may be administered by implantation (for example subcutaneously or intramuscularly) or by intramuscular injection. Thus, for example, the compounds can be formulated with suitable polymeric or hydrophobic materials (for example as an emulsion in an acceptable oil) or ion exchange resins, or as sparingly soluble derivatives, for example, as a sparingly soluble salt.

Because the action of a neuroprotectant composition is in the central nervous system, delivery techniques can be designed to permit the composition to cross the blood-brain barrier. Such techniques are known in the art (for example, see PCT WO 89/10134, Cloughesy and Black, J. Neurooncol., 26:125-132, 1995; and Begley, J. Pharm. Pharmacol., 48:136-146, 1996, all of which are incorporated herein in their entirety). Components of a neuroprotectant composition can also be modified (e.g., chemically) using methods known in the art to facilitate their entry into the CNS.

In some cases, it may be desirable to deliver a neuroprotectant composition directly to the nervous system, especially when one or more components of a neuroprotectant composition do not cross the blood-brain barrier. Examples of such methods are intraventricular injection (Kordower et al., Exp. Neurol., 124:21-30, 1993) or installation of an osmotic pump (e.g., an Alzet® pump). Another example of such a method is to surgically place an Omaya reservoir-shunt with in-line filter into the cisternal space. Neuroprotectant composition in an appropriate excipient (e.g., phosphate-buffered saline) is instilled into the shunt by injection on a prescribed basis. In all cases, consideration is given to the appropriate formulation used for specific forms of delivery.

For administration by inhalation, a neuroprotectant composition is delivered, for example, as an aerosol spray with the use of a suitable propellant, for example, dichlorodifluoromethane, trichlorofluoromethane, dichlorotetrafluoroethane, carbon dioxide, or other suitable gas. Other suitable methods of nasal delivery known in the art can be used, including those that facilitate delivery of a predetermined dosage.

The compositions can, if desired, be presented in a pack or dispenser device, which may contain one or more unit dosage forms containing the active ingredient. The pack may for example comprise metal or plastic foil, such as a blister pack. The pack or dispenser device can be accompanied by instructions for administration.

The therapeutic compositions of the invention can also contain a carrier or excipient, many of which are known to skilled artisans. Excipients that can be used include buffers (for example, citrate buffer, phosphate buffer, acetate buffer, and bicarbonate buffer), amino acids, urea, alcohols, ascorbic acid, phospholipids, proteins (for example, serum albumin), EDTA, sodium chloride, liposomes, mannitol, sorbitol, and glycerol.

Methods for making such formulations are well known and can be found in, for example, “Remington's Pharmaceutical Sciences, Gennaro, A. R. ed. 20^{th} edition, 2000, Williams & Wilkins PA, USA”.

Typically, the dosage for intravenous administration will be greater than that for intracisternal administration, e.g., 10 to 1,000 μg/kg of an HN derivative may be administered. For example, the derivatives may be administered intravenously at concentrations ranging from 1-100 μg/kg/hour.

The target diseases to be prevented or treated using an HN derivative of the present invention, or using a vector that expresses the HN derivative is not limited in any way, so long as the used the HN derivative is effective for treating the disease. Examples of preferred target diseases include neurodegenerative disorders, in particular Alzheirner's disease. Previous studies have revealed that cell death of neurons occurs in Alzheimer's disease (I. Nishimoto et al., 1997, Adv. Pharmacol., 41: 337-368). Some sort of activation of APP (I. Nishimoto et al., 1998, Neurobiol. Aging., 19: S33-S38) and presenilin (Nishimura et al., 1999, Clin. Genet. 55: 219-225) are suggested to be associated with the cell death. Therefore, pharmaceutical compositions of this invention are expected to be applicable as medicament for protection against neurodegeneration that occurs in Alzheiiner's disease. In addition to Alzheimer's disease, for example, diseases caused by cell death of neurons due to cerebral ischemia (T. Kirino, 1982, Brain Res., 239: 57-69) can be prevented by the use of a pharmaceutical composition of the present invention. Further, Parkinson's disease that accompanies dementia (M. H. Polymeropoulos et al., 1997, Science, 276: 2045-2047); diffuse Lewy bodies disease (M. G. Spillantini et al., 1998, Proc. Natl. Acad. Sci. USA, 95: 6469-6473); dementia that accompanies Down's disease; and such are also targets of the treatment and prevention using a protein of the invention. Furthermore, since. APLP1, which is an APP analogue, is said to be the causative gene for congenital nephrotic syndrome (Lenkkeri, U. et al., 1998, Hum. Genet. 102: 192-196), renal diseases, such as nephrotic syndrome, is also the target for the treatment and prevention.

### BEST MODE FOR CARRYING OUT THE INVENTION

The invention is further described in the following examples, which do not limit the scope of the invention described in the claims.

### EXAMLES

In the examples below, HN derivatives or combinations of them, such as those listed in Table 1, are shown to inhibit the cell death normally caused by FAD gene mutants or Aβ peptides, as measured by cell death assays that monitor the cell mortality present in cell culture. Further, the examples demonstrate that these HN derivatives inhibit the cell death caused by Aβ peptides and FAD gene mutants, at least in part, by forming multimers.

In the examples below, the following materials and general methodologies are used throughout, unless otherwise specified.

TABLE 2
List of peptides used in the examples

| | | |
|---|---|---|
| Peptides | SEQ ID NO: | Sequence |

| | | |
|---|---|---|
| HN | 1 | MAPRGFSCLLLLTSEIDLPVKRRA |
| HNG | 2 | MAPRGFSCLLLLTGEIDLPVKRRA |
| HNA | 3 | MAPRGFSALLLLTSEIDLPVKRRA |
| P-S14 HN | 4 | MAPRGFSCLLLLT(p-S)EIDLPVKRRA |
| P-S7 HN | 5 | MAPRGF(p-S)CLLLLTSEIDLPVKRRA |
| P-S7/14 HN | 6 | MAPRGF(p-S)CLLLLT(p-S)EIDLPVKRRA |
| D-Ser14 HN | 7 | MAPRGFSCLLLLT(D-S)EIDLPVKRRA |
| D-Ser7 HN | 8 | MAPRGF(D-Ser)CLLLLTSEIDLPVKRRA |
| D-Ser7/14 HN | 9 | MAPRGF(D-Ser)CLLLLT(D-Ser)EIDPPVKRRA |
| AGA-(D-Ser14) HN | 10 | MAPAGASCLLLLT(D-Ser)EIDLPVKRRA |
| AGA-(D-Ser14) HN17 | 11 | PAGASCLLLLT(D-Ser)EIDLP |
| S7A HN | 12 | MAPRGFACLLLLTSEIDLPVKRRA |
| S7A HNG17 | 13 | PRGFACLLLLTSEIDLP |
| HNG-KKK | 14 | YMAPRGFSCLLLLTGEIDLPVKKKK |
| EF-(S7A)HN | 15 | EFLIVIKSMAPRGFACLLLLTSEIDLPVKRRA |
| EF-HNG-KKK | 16 | EFLIVIKSMAPRGFSCLLLLTGEIDLPVKKKK |
| EF-HN | 17 | EFLIVIKSMAPRGFSCLLLLTSEIDLPVKRRA |
| EH-HNA | 18 | EFLIVIKSMAPRGFSALLLLTSEIDLPVKRRA |
| EF-HNG | 19 | EFLIVIKSMAPRGFSCLLLLTGEIDLPVKRRA |
| EFLIVIKS | 20 | EFLIVIKS |
| AGA-HNG | 21 | MAPAGASCLLLLTGEIDLPVKRRA |
| EF-AGA-HNG | 22 | EFLIVIKSMAPAGASCLLLLTGEIDLPVKRRA |
| HNG-17 | 23 | PRGFSCLLLLTGEIDLP |

Note:
Amino acid residues not present in a naturally occurring HN are in bold face font. “p-S” represents phosphorylated L-Ser.

Genes, Polypeptides, and Materials

V642I-APP, NL-APP, M146L-PS1, and N141I-PS2 cDNAs, all in CMV promoter-driven pcDNA vectors, were described previously [2, 14, 15, 21]. HN and HNG were purchased from Peptide Institute (Minoh, Osakca, Japan). Other HN peptides were chemically synthesized. See the “Methods of Making the New HN Derivatives” section of this application for more detail. The compositions of the synthesized peptides were confirmed by reverse phase HPLC. Aβ1-43 peptide was purchased from Peptide Institute. Other materials were from commercial sources.

Cells and Cell Death Assays

In the disclosure below, several cell-based assays used to illustrate that small peptides inhibit a neuronal cell from cytotoxicity are described.

In one assay, transfection with V642I-APP causes cell death in F11 neuronal hybrid cells [2, 12, 14, 15]. In the other, treatment with Aβ peptides causes cell death in primary cultured cortical neurons [14-18, 24-26].

F11 cells, the hybrid of a rat embryonic-day-13 primary cultured primary neuron and a mouse neuroblastoma cell, are one of the best models for primary cultured neurons as they carry representative characteristics of primary neurons, such as generation of action potentials without differentiation by NGF [22]. They have been used in various studies of is neuronal functions [2, 11-16, 26-31]. Using these cells, the underlying mechanisms for cytotoxicity by V642I-APP have well been analyzed [13]. Cytotoxicity by V642 mutants of APP has also been confirmed in multiple different systems [3, 6, 10, 21]. F11 cells were grown in Ham's F-12 (GibcoBRL, Gaithersburg, Md.) plus 18% fetal bovine serum (FBS; Hyclone, Logan, Utah) and antibiotics, as described previously [16]. F11 cells (7×10⁴ cell/well in a 6-well plate cultured in Ham's F-12 plus 18% FBS for 12-16 hr) were transfected with plasmids encoding FAD genes by lipofection [FAD cDNA 2 μg, LipofectAMINE 4 μl, PLUS reagent 8 μl (GibcoBRL)] in the absence of serum for 3 hr, and were incubated with Ham's F-12 plus 18% FBS for 2 hr. Then, culture media were changed to Ham's F-12 plus 10% FBS with or without HN peptides, and cells were cultured for an additional 67 hr. Seventy-two hr after transfection, cell mortality was measured by Trypan blue exclusion assay, performed as follows. Cells were suspended by pipetting gently, and 50 μl of 0.4% (finally 0.08%) Trypan blue solution (Sigma, St Louis, Mo.) was mixed with 200 μl of the cell suspension at room temperature. Stained cells were counted within 3 min after the mixture with Trypan blue solution.

The primary culture of mouse cortical neurons was performed in poly-L-lysine-coated 24-well plates or 96-well plates (Sumitomo Bakelite, Akita, Japan), in the absence of serum and the presence of N2 supplement, as described previously [16]. The purity of neurons by this method was >98%. Prepared neurons (for neurons seeded in 96-well plates: 2.5×10⁴ or 5×10⁴ cells/well, 100 μl culture medium/well; for neurons seeded in 24-well plates: 1.25×10⁵ cells/well, 250 μl culture medium/well) were preincubated with or without 10 μM (in case for the experiment with 10 μM HN) synthetic HN for 16 hr, and treated with 25 μM Aβ in the presence or absence of 10 μM HN for 72 hr, respectively. As primary neurons were vulnerable to transient dryness during medium exchange, we treated neurons with 25 μM Aβ as follows. First, a half volume (50 μl for neurons in 96-well plates; 125 μl for neurons in 24-well plates) of old medium was discarded. Then, the same volume (50 μl for neurons in 96-well plates; 125 μl for neurons in 24-well plates) of prewarned fresh medium containing 10 μM HN and 50 μM Aβ (Aβ was directly resolved) was added to the culture. The Aβ peptides used at 25 μM to induce neurotoxicity formed aggregation in the medium during the cell culture at 37° C. for 72 hr. as described [14, 15]. The WST-8 cell viability assay was performed, using Cell Counting Kit-8 (Wako Pure Chemical Industries, Osaka, Japan), as described previously [14-16, 23].

Calcein assay was performed with Calcein-AM {3′,6′-di-(O-acetyl)-2′,7′-bis [N,N-bis(carboxymethyl)aminomethyl] fluorescein, tetraacetoxymethyl ester; Dojindo}, as described previously [14, 16]. These assays were simultaneously performed in the experiments with neurons seeded in 96-well plates, as follows. Seventy-two hours after Aβ treatment, cells were added with the mixture of 10 μl WST-8 solution and 1 μl of 600 μM Calcein-AM. After 2 hr incubation in a CO₂ incubator at 37° C., WST-8 absorbance was measured as OD₄₅₀. After exchanging the medium to PBS to lower the background, Calcein-specific fluorescence (ex=485 nm, em=535 nm) was observed by fluorescence microscopy or measured by a spectrofluorometer (Wallac1420 ARVOsx Multi Label Counter). Most cell viability data using Calcein assay and WST-8 assay were obtained from neurons seeded at 5×10⁴ cells/well in 96-well plates (high-density neurons) and confirmed by both assays with the cells seeded at 2.5×10⁴ cells/well (low-density neurons).

Some experiments for Calcein assay were performed with neurons seeded in 24-well plates. In these experiments, the appropriate setting of the spectrofluorometer for Calcein fluorescence measurement (the scanning time=0.5 sec) yielded apparently high background, as shown in FIGS. 5 and 6, relative to the background in Calcein fluorescence measurement of 96-well plates (the scanning time=1 see). In most experiments using primary neurons, four independent experiments were performed and the means? S. D. of the results were discussed.

Immunoblot Analysis

Immunoblot analysis was performed as described [14-16]. For the analysis of transfected FAD gene expression, lysates (20 μg/lane) from cells transfected with each FAD gene (V642I-APP, NL-APP, M146L-PS1, N141I-PS2) were submitted to SDS-PAGE and were electrically blotted to a PVDF sheet. After blocking, the sheet was soaked with antibodies [for APP mutants: 5 μg/ml 22C11 (CHEMICON, Temecula, Calif.); for PS1 mutant: 1/2000 of anti-PS1 N-terminus antibody (CHEMICON) for PS2 mutant: 1/500 of anti-PS2 antibody (Cell Signaling Technology, Beverly, Mass.)] and then with HRP-labeled secondary antibodies. The antigenic bands were visualized by ECL (Amersham Pharmacia Biotech, Uppsala, Sweden).

Dimerization Experiment

Synthetic (His)₆-tagged HN derivative peptides (1 nmol) was incubated with 20 μl of Ni-NTA agarose (QIAGEN, Hilden, Germany) in a total volume of 0.5 ml of buffer A [20 mM Tris/HCl (pH8.0) and 1 mM DTT] for 1 hr at 4° C. and washed once with buffer B [20 mM Tris/HCl (pH 7.4), 1 mM DTT, and 150 mM NaCl]. Synthetic FLAG-tagged peptides (1 nmol) were mixed with the beads immobilizing (His)₆-tagged peptides in a total volume of 0.5 ml of buffer B for 2 hr at 4° C. on a rotating shaker, and washed three times with buffer B. The washed beads were mixed with 20 μl of 2× sampling buffer [100 mM Tris/HCl (pH6.8), 200 mM DTT, 4% (w/v) SDS, 0.2% bromophenol blue, and 20% (v/v) glycerol] and boiled. The supernatant was submitted to Tris/Tricine SDS-PAGE, followed by immunoblotting analysis with 1/5000 anti-FLAG monoclonal antibody M2 (Sigma).

Statistical Analysis

All experiments described herein were repeated at least three times with independent transfections, treatments, or precipitations, each of which yielded similar results. Statistical analysis was performed with one-way ANOVA followed by post-hoc test, in which p<0.05 was assessed as significant.

### Example 1

Cell death assays were carried out to illustrate that phospho-Ser HN peptides or HN peptides inhibit a neuronal cell from cytotoxicity. The results in Tables 34 show that each of P-S14 HN(SEQ ID NO: 4), P-S7 HN(SEQ ID NO: 5), or P-S7/14 HN (SEQ ID NO: 6) exerted neuronal cytoprotection against the Aβ treatment or V642I-APP in dose-response curves similar to the dose-response curves of the neuroprotective action of authentic HN (SEQ ID NO: 1). Both the action potency and efficacy of these phosphorylated HN derivatives were similar to those of HN.

Table 3 shows cell viability of primary neurons that were treated with 25 μM Aβ1-43 and cultured with or without phospho-Ser HN peptides (SEQ ID NOs: 4-6) or HN (SEQ ID NO: 1) for 72 hours. Values in Table 3 represent the percentage (means±S.D) of Calcein fluorescence from neurons without treatment.

Table 4 shows cell viability of F11 neuronal hybrid cells that were treated with pcDNA (vec) or V642I-APP cDNA and cultures with or without phospho-Ser HN peptides (SEQ ID NOs: 4-6) or HN (SEQ ID NO: 1). Cell viability measured 72 hr after the onset of transfection by Trypan blue exclusion. “no-T” means no transfection. In these experiments, expression of V642I-APP was not affected by HN peptides. Values shown in Table 4 are indicated as the % (means±S.D) dead cells of total cells. In Experiment 1 and all of following experiments, CMV-promoter driven expression of V642I-APP, NL-APP, M146L-PS1, or N141I-PS2 holoprotein was examined and found not affected by peptides tested.

TABLE 3

| | | | Aβ + peptide | | | | | | |
|---|---|---|---|---|---|---|---|---|---|
| | None | Aβ | 10 pM | 100 pM | 1 nM | 10 nM | l00 nM | 1 μM | 10 μM |
| None | 100 ± 5.3^{%} | 8.9 ± 0.8 | | | | | | | |
| P-S7HN | | | 9.0 ± 0.9 | 8.7 ± 0.8 | 8.8 ± 0.9 | 8.8 ± 0.6 | 10.5 ± 0.9 | 84.2 ± 6.8* | 103.3 ± 2.8* |
| P-S14HN | | | 8.6 ± 0.3 | 8.6 ± 0.4 | 8.9 ± 0.1 | 8.6 ± 0.3 | 8.6 ± 0.1 | 85.8 ± 4.1* | 104.1 ± 1.7* |
| P-S7/14 HN | | | 9.3 ± 0.4 | 8.8 ± 0.3 | 8.8 ± 0.2 | 8.7 ± 0.3 | 8.6 ± 0.2 | 84.0 ± 5.8* | 103.3 ± 0.8* |
| HN | | | | | | 7.5 ± 1.0 | 13.8 ± 1.3 | 78.9 ± 4.4* | 91.6 ± 7.6* |

(*p < 0.01 vs Aβ treated cells)

TABLE 4

| | | | | V6421-APP + peptide | | | | | | |
|---|---|---|---|---|---|---|---|---|---|---|
| | No T | Vec | V6421-APP | 10 pM | 100 pM | 1 nM | 10 nM | 100 nM | 1 μM | 10 μM |
| None | 9.3 ± 1.2^{%} | 9.7 ± 1.4 | 52.9 ± 1.1 | | | | | | | |
| P-S7HN | | | | 53.7 ± 0.8 | 53.1 ± 1.7 | 54.8 ± 1.6 | 54.1 ± 1.4 | 403 ± 1.5* | 220 ± 2.7* | 11.6 ± 1.1* |
| P-S14HN | | | | 56.5 ± 4.1 | 56.3 ± 2.7 | 54.3 ± 1.2 | 53.4 ± 2.0 | 41.8 ± 1.8* | 222 ± 2.4* | 121 ± 0.5* |
| P-S7/14 HN | | | | 54.8 ± 0.5 | 54.1 ± 1.5 | 52.9 ± 1.6 | 53.8 ± 3.1 | 38.2 ± 1.9* | 22.7 ± 5.2* | 11.8 ± 1.2* |
| HN | | | | | | | 53.3 ± 0.9 | 29.6 ± 1.2* | 17.1 ± 2.1* | 10.3 ± 0.5* |

(*p < 0.1 vs cells transfected with V6421-APP cDNA alone)

### Example 2

In Experiment 2, cell death assay results illustrate the ability of HN derivative with amino acid substitution at Ser¹⁴ to protect against V642I-APP-induced cell death. Five representative amino acids were examined. They are Arg (a representative of basic residues), Glu (a representative of acidic residues), Trp (a representative of aromatic residues), Thr (a representative of OH-containing residues), and Pro (a unique residue). When the basal cytotoxicity without transfection or induced by an empty pcDNA vector was 9.0±1.4 or 10.0±1.9 (in % dead cells of total cells), respectively, and the cytotoxicity by V642I-APP transfection was 53.7±1.2%, the cytotoxicity by V642I-APP in the presence of (S14R)HN, (S14W)HN, (S14T)HN, or (S14E)HN (each 10 μM) was 52.6±1.0%, 54.5±4.2%, 54.0±2.3%, or 45.0±1.9%, respectively. Under the same conditions, however, the cytotoxicity by V642I-APP in the presence of 10 μM (S14P)HN (SEQ ID NO: 4) or authentic HN (SEQ ID NO: 1) was 12.5±1.0% or 11.0±0.1%, respectively. These data indicate that Pro functionally substitutes for Ser¹⁴ in regard to suppression of V642I-APP-induced neuronal cell death.

### Example 3

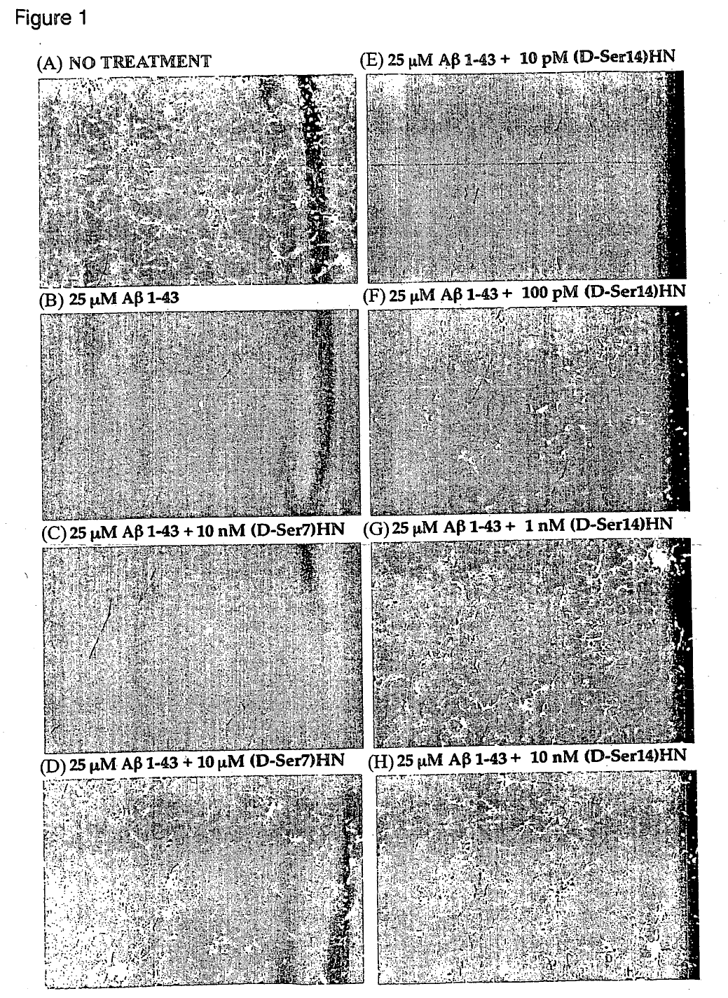

This experiment was carried out to illustrate the ability of HN derivatives with D-Ser substitution to protect against neuronal cell death. The derivatives include (D-Ser¹⁴)HN , (D-Ser⁷)HN and (D-Ser^{7/14})HN (SEQ ID NOs: 7-9). The present inventors considered the meaning of the result that only Gly and Pro can functionally replace Ser¹⁴ among various kinds of residues. Gly is the sole amino acid that has no D-form, as it lacks a side chain. Pro has a side chain whose molecular space is small. They therefore hypothesized that a small or no side chain of Pro or Gly, respectively, might be advantageous in maintaining an active conformation of HN, which could potentially be realized by D-racemation of Ser¹⁴. As shown in FIG. 1 (microscopic views of Calcein-stained neurons) and 2-4 (quantitative assays), (D-Ser¹⁴)HN suppressed neuronal death by 25 μM Aβ1-43 with IC₅₀ of 100 pM-1 nM and exerted full neuroprotection at 1-10 nM, as assessed with Calcein fluorescence assay and WST-8 absorbance assay as well as Trypan blue exclusion assay (data not shown). In contrast, authentic HN exerted full suppression at 10 μM with IC₅₀ of 100 nM-1 μM (see FIGS. 12-14). These data indicate that D-racemation of Ser at position 14 allowed HN to exert neuroprotection against Aβ with 100-1000-times as high potency as authentic HN. Similarly, (D-Ser¹⁴)HN exerted potent cytoprotection against V642I-APP as well as other FAD mutants (NL-APP, M146L-PS1, N141I-PS2). It has been established that NL-APP, M146L-PS1, or N141I-PS2 causes cytotoxicity in F11 neuronal hybrid cells [13-16], and the underlying mechanisms for cytotoxicity by these FAD mutants have well been analyzed [13, 17, 18]. All IC₅₀values were commonly between 100 pM and 1 nM, and full suppression was caused by 10 nM (D-Ser¹⁴)HN. This potency of action was higher by two to three orders of magnitude than that of HN and virtually identical to that of HNG. (D-Ser¹⁴)HN did not suppress CMV-promoter-driven expression of either V642I-APP, NL-APP, M146L-PS1, or N141I-PS2 holoprotein (data not shown).

In contrast, D-isomer replacement of Ser at position 7 did not alter the potency and efficacy of neuroprotective action of HN. (D-Ser⁷)HN exhibited action potency similar to that of HN against neuronal cell death by Aβ1-43, V642I-APP, NL-APP, M146L-PS1, or N141I-PS2. Also, replacement of D-Ser⁷ did not affect the neuroprotective effect of (D-Ser¹⁴)HN. The cytoprotective actions of HN with both D Ser⁷- and D-Ser¹⁴ [(D-Ser^{7/14})HN against V642I-APP, NL-APP, M146L-PS1, or N141I-PS2 were virtually identical to those of (D-Ser¹⁴)HN. Taken together, these data indicate that the action of HN is not augmented by D-racemation at Ser⁷ and that HN is specifically activated by D-Ser racemation at position 14.

### Example 4

The inventors and others [16] previously reported that Ala substitution at both Arg⁴ and Phe⁶ further augments the potency of HNG 3-10 times and that (R4A/F6A)HNG (AGA-HNG) exerts full neuroprotection at 100-300 pM. The potentiation by R4A/F6A substitution of HNG action was attributed to a loss of trypsin/chemotrypsin cleavage sites and protection of HNG from proteases. In the experiment, effects of AGA-(D-Ser¹⁴)HN (SEQ ID NO: 10) and AGA-(D Ser¹⁴)HN17(SEQ ID NO: 11) on cytoprotection were examined. AGA-(D-Ser¹⁴)HN (SEQ ID NO: 10) exerted full cytoprotection at 100 pM against each of V642I-APP, NL-APP, M146L-PS1, N141I-PS2, or Aβ1-43 (FIGS. 5-6). The IC₅₀ values of its cytoprotective actions were commonly about 10 pM. In these experiments, CMV-promoter-driven expression of either V642I-APP, NL-APP, M146L-PS1, or N141I-PS2 holoprotein was not affected by AGA-(D-Ser¹⁴)HN (data not shown).

As disclosed in [16], the Pro³-Pro¹⁹ region (PRGFSCLLLLTSEIDLP) (HN17, SEQ ID NO: 25) of HN is the essential core domain with inhibitory activity of full-length HN, and the synthetic peptide for the Pro³-Pro¹⁹ domain (HN17, SEQ ID NO: 25) is as potent in cytoprotection as authentic HN. In this experiment, the effects of D-Ser¹⁴-containing Pro³-Pro¹⁹ with R4A/F6A [PAGASCLLLLT(D-Ser)EIDLP] (SEQ ID NO: 11), termed AGA-(D-Ser¹⁴)HN17, on neuronal cell death by V642I-APP, NL-APP, M146L-PS1, or N141I-PS2 were also examined. AGA-(D-Ser¹⁴)HN17 (SEQ ID NO: 11) dose-dependently suppressed neuronal cell death by each of these FAD genes with IC₅₀ at about 10 pM and fully at 100 pM. AGA-(D-Ser¹⁴)HN17 did not suppress CMV-promoter-driven expression of either V642I-APP, NL-APP, M146L-PS1, or N141I-PS2 holoprotein (data not shown).

Therefore, AGA-(D-Ser¹⁴)HN17 (SEQ ID NO: 11) is as potent as AGAD-Ser¹⁴)HN in neuroprotection.

### Examples 5, 6, and 7—Multimerization

In Examples 5, 6 and 7 (FIGS. 10-14), it was shown that an N-terminal fusing with EFLIVIKS (SEQ ID NO: 20) or EFLIVKS (SEQ ID NO: 24), which is the established amino acid sequence to form a dimer [32, 33], restored the ability of some HN derivatives to suppress neuronal cell death.

In Example 5, it was indicated that, at up to 10 μM, (S7A)HN (SEQ ID NO: 12) exerted no suppression of neuronal death by Aβ1-43 in primary cultured neurons (FIGS. 7-8). Two independent cell viability assays with low (FIG. 8A, 8B) and high (FIG. 8C, 8D) densities of primary neuron cultures indicated quite similar results pointing to no effect of (S7A)HN. This was also the case with the effect of (S7A)HN on neuronal cytotoxicity by V642I-APP, NL-APP, M146L-PS1, or N141I-PS2 (FIG. 9A for V642I-APP; data not shown for other FAD genes). Lack of the cytoprotective action of (S7A)HN was confirmed by using a plasmid coding for this mutant peptide (pHN/S7A). F11 neuronal cell death by V642I-APP was not suppressed by co-transfection with pHN/S7A, as was the case with a plasmid coding for (C8A)HN (pHNA), whereas transfection with the plasmid coding for HN or HNG (pHN or pHNG) resulted in significant suppression of neuronal cytotoxicity by V642I-APP [basal cytotoxicity without transfection (% dead cells, means±S. D.): 10.2±0.6%; cytotoxicity by pFLAG (the empty plasmid for pHN): 9.5±0.5%; cytotoxicity by V642]-APP plus pFLAG: 55.2±1.3%; cytotoxicity by V642I-APP plus pHN: 12.0±2.2%; cytotoxicity by V642I-APP plus pHNG: 11.3±0.7%; cytotoxicity by V642I-APP plus pHNA: 55.9±1.0%; cytotoxicity by V642I-APP plus pHN/S7A: 55.8±2.7%]. In this experiment, expression of V642I-APP was not affected by co-transfection of each plasmid coding for HN or HN derivatives (data not shown), as reported previously [14]. Therefore, Ser⁷ was essential in order for HN to exert cytoprotection. This residue thus plays an essential role but in a manner different from that of Ser¹⁴.

In contrast, (S7A)HNG17 exerted suppressive effects on neuronal death by Aβ1-43 in primary neurons cultured with IC₅₀ of about 1 nM, and its full suppression was observed at about 10 nM (FIGS. 7-8). This was also the case with F11 neuronal cytotoxicity caused by V642I-APP (FIG. 9A). These data indicate that (S7A)HN has no neuroprotective action, although the shorter (S7A)HNG17 exerts potent neuroprotective action.

In Example 6 (FIGS. 7 and 9B), it was shown that HNG with its C-terminal KRRA substituted to KKKK (HNG-KKK, SEQ ID NO: 14) lost the neuroprotective action (microscopic views of the 10 nM effect and the 100 μM effect in FIGS. 7 and 10, respectively; Calcein and WST-8 assay data in FIGS. 9B and 12-13). This is unexpected because the cytoprotective action of full-length HN was previously found only marginally affected by the substitution of the C-terminal KRRA to KAAA or by the deletion of the C-terminal VKRRA [14, 16]. Based on these observations, it is likely that at least two different steps would be necessary for HN to exert its cytoprotective action. One is the binding of HN to its putative receptor on the cell surface, as is supported by the presence of a specific binding site on the surface of neuronal cells [14]. Another is a process through which the C-terminal KKK substitution nullifies the action of the Pro³-Pro¹⁹ core domain. This process may be a multimerization of HN.

In Example 7 (FIGS. 10-14), it was indicated that multimerization of HN peptides restored the neuroprotectivity of (S7A)HN and HNG-KKK.

The HNG-KKK with an N-terminal fusing with EFLIVIKS (EF) was synthesized in order to investigate the effect of dimerization. In primary neurons treated with 25 μM Aβ1-43, EF-HNG-KKK (SEQ ID NO: 16) exerted full neuroprotection at 10-100 nM with the IC₅₀ value of about 1 nM, whereas non-fused HNG-KKK elicited no action even at 10 μM (FIGS. 10-11, and right panels in FIGS. 12-13). EFLIVIKS alone exerted no neuroprotection at up to 100 μM in the same system (FIG. 10 and left panels in FIGS. 12-13). These findings were confirmed for the action of EF-HNG-KKK against neuronal cytotoxicity by V642I-APP in F11 cell system. EF-HNG-KKK potently exerted cytoprotection at low nM concentrations, whereas HNG-KKK had no effect even at 100 μM in the same system (FIG. 10). EFLIVIKS alone exerted no cytoprotection at up to 100 μM (FIGS. 12-14, left panel). The IC₅₀ value of the EF-HNG-KKK action was about 100 pM and complete suppression was achieved by 10 nM EF-HNG-KKK. Taking all these data together, it was very likely that the C-terminal KKK precluded HNG from potential dimerization and that this inhibition was further antagonized by the fused dimerization tag.

Based on the results from HNG-KKK and EF-HNG-KKK, it is likely that HN17 is highly prone to dimerize due to lack of the C-terminal residues if Ser⁷ is required for authentic HN, bearing the C-terminal KRRA, to dimerize at low μM, but is not necessary for HN17 to dimerize, the EFLIVIKS fusion would allow (S7A)HN to exert cytoprotection, because (S7A)HNG17 was as effective as HNG17, as noted above. Conversely, if the EFLIVIKS fusion does not allow (S7A)HN to exert cytoprotection, dimerization would not be a necessary process for the HN action.

As expected, EFLIVIKS-fused (S7A)HN [abbreviated as EF-(S7A)HN, SEQ ID NO: 15] protected against the cytotoxicity by Aβ1-43 in primary neurons (FIGS. 10-11 and leftmost panels in FIGS. 12-13). EF-(S7A)HN exerted full suppression of Aβ neurotoxicity at 100 nM with the IC₅₀ value of about 10 nM, although neither (S7A)HN nor EFLIVIKS alone exerted any neuroprotection at all. EFLIVIKS-HN (EF-HN, SEQ ID NO: 17) also exerted neuroprotection with potency about 100 times higher than that of HN, that is, potency virtually identical to that of EF-(S7A)HN against Aβ neurotoxicity (FIGS. 10-11 and second panels from left in FIGS. 12-13). In contrast, (C8A)HN(HNA) (MAPRGFSALLLLTSEIDLPVKRRA/SEQ ID NO: 3) was completely inactive, even when it was fused with EFLIVIKS (FIG. 11 and third panels from left in FIGS. 12 and 13).

In V642I-APP-expressing F11 neuronal cells, EF-(S7A)HN also exerted cytoprotection with IC₅₀ at 1 nM and fully at 100 nM (FIG. 14). This potency was about 100 times higher than that of HN. Under the same conditions, (S7A)HN exerted no cytoprotection at up to 10 μM. EF-HN exerted cytoprotection with potency about 100 times higher than that of HN, that is, potency equivalent to that of EF-(S7A)HN (second panel from leftin FIG. 14). In contrast, (C8A)HN was again completely inactive, even when it was fused with EFLIVIKS (third panel from left in FIG. 14).

### Example 8

Results from Experiment 8 (FIGS. 15 and 16) suggested a blocking effect of EFLIVIKS on the rescue actions of EF-(S7A)HN and EF-HNG-KKK. If neuroprotection by EF-(S7A)HN or EF-HNG-KKK is through dimerization of fused EFLIVIKS, their action would be blocked by adding an excess amount of isolated EFLIVIKS peptide, because the isolated EFLFVIKS peptide would bind to the EFLIVIKS region of EF-(S7A)HN or EF-HNG-KKK and keep the (S7A)HN region or the HNG-KKK region in a monomeric status. Conversely, if fused EFLIVIKS plays a role different from dimerization, such as stabilization of the fused peptide, addition of EFLIVIKS would not inhibit the rescue action of EF-(S7A)HN or EF-HNG-KKK. As shown in FIGS. 15-16, while massive neuronal death by 25 μM Aβ1-43 was dose-dependently suppressed by EF-(S7A)HN or EF-HNG-KKK in the absence of EFLIVIKS, EF-(S7A)HN or EF-HNG-KKK could not suppress Aβ neurotoxicity at all in the presence of 100 μM EFLIVIKS. In clear contrast, 10 μM EF-HN or EFLIVIKS-HNG (EF-HNG) could fully suppress Aβ neurotoxicity even in the presence of 100 μM EFLIVIKS. This result is consistent with the notion that HN or HNG at 10 μM is able to exert full neuroprotection through dimerization of HN or HNG per se. Essentially the same results were obtained from two different viability assays (FIG. 15). These data strongly indicate that EF-(S7A)HN or EF-HNG-KKK exerts neuroprotective function by self-dimerization through the fused EFLIVIKS.

### Example 9

In this example (FIG. 17), it was proved that positive or negative dimerization of HN and other HN derivatives exist. Synthetic (His)₆-HN was mixed with synthetic HN-FLAG, and precipitated by Ni-beads. The precipitate was examined with an anti-FLAG antibody for HN-FLAG. As shown in FIG. 17A, HN-FLAG was precipitated by (His)₆-HN, while Ni-beads by themselves were not able to precipitate HN-FLAG (data not shown). Under the same conditions, (S7A)HN-FLAG was not precipitated by (His)₆-(S7A)HN. EF-(S7A)HN-FLAG was precipitated by (His)₆-EF-(S7A)HN as efficiently as HN-FLAG precipitation by (His)₆-HN. In the Tris/Tricine gels used, the migration of EF-(S7A)HN-FLAG little altered from that of HN, but, instead, its migration sometimes resulted in a broad band due to insufficient stacking.

To confirm that HN-FLAG was precipitated by (His)₆-HN through their HN regions, tag-free HN was added (FIG. 17B). In the presence of excess amounts of tag-free FIN, little amounts of HN-FLAG were precipitated by (His)₆-HN, whereas excess amounts of EFLIVIKS could not interfere HN-FLAG precipitation by (His)₆-HN. In contrast, EF-(S7A)HN-FLAG precipitation by (His)₆-EF-(S7A)HN was blocked by adding excess amounts of EFLIVIKS, but not (S7A)HN. It was also found that HNG-KKK had no effect on HN-FLAG precipitation by (His)₆-HN under the condition in which the same concentration of HNG abolished the HN-FLAG/(His)₆-HN interaction. In each experiment shown in FIG. 17, (His)₆-tagged HN peptides were similarly precipitated (data not shown). These data confirm that (i) HN can self-dimerize; (ii) (S7A)HN has little activity for self-dimerization: (iii) EFLIVIKS fusion allows (S7A)HN to dimerize; and (iv) HNG-KKK has little activity to form a complex with HN.

### Example 10

In this example (FIGS. 18-21), more potent derivatives of HN were analyzed. For example, EFLIVIKS-fused HNG (EF-HNG, SEQ ID NO: 19) exerted full cytoprotection at 1 nM with IC₅₀ of about 100 pM, as assessed by both Calcein fluorescence and WST-8 absorbance assays (upper and lower panels in FIG. 19, respectively). As the IC₅₀ value of the HNG action against Aβ1-43-induced neuronal death was about 500 pM, as described previously [16], the fusion with EFLIVIKS resulted in a few times further potentiation of the HNG action. Four panels in FIG. 20 indicates that EF-HNG exerted cytoprotection against neuronal cytotoxicity by either V642I-APP, NL-APP, M146L-PS1, or N141I-PS2 with IC₅₀ of about 10 pM, whereas the authentic HNG suppressed cell death caused by the FAD genes with IC₅₀ of about 30 pM, indicating, again, that EFLIVIKS fusion potentiates the action of HNG by a factor of two to three.

Even more potent than EF-HNG, EF-AGA-HNG (SEQ ID NO: 22) exerted neuroprotection against 25 μM Aβ1-43 at 10 pM (FIG. 18 and upper and lower panels in FIG. 19). The IC₅₀ values were about 100 fM, as assessed by Calcein assay, and about 1 pM, as assessed by WST-8 assay. The observed difference in the IC₅₀ values between these two assays was attributed to the well-known fact that Calcein fluorescence of neurons reflects not only viability of neurites but also viability of neuronal bodies, whereas WST-8 assay mainly represents viability of neuronal bodies. Therefore, the result indicates that this HN derivative has a slightly stronger action on neuritic viability than on neuronal body viability.

The enhanced potency of the cytoprotective action of EF-AGA-HNG was also the case with neuronal cytotoxicity by FAD genes. As shown in the right four panels in FIG. 20, at 10 pM, EF-AGA-HNG exerted full cytoprotection against neuronal cytotoxicity by either V642I-APP, NL-APP, M146L-PS1, or N141I-PS2. The IC₅₀ values were commonly about 100 fM. The observed difference in the action potency between EF-HNG and AGA-HNG was not observed in F11 cells. Treatment of FAD-gene-transfected F11 cells with various concentrations of AGA-HNG or EF-AGA-HNG resulted in little or only marginal alteration in the expression of each FAD gene (FIG. 21). EF-AGA-HNG is thus the most potent HN derivative thus far known to exert cytoprotection against neuronal cytotoxicity by FAD genes.

List of the HN Derivatives and Other Peptides (Table 5)

| | | Effect | | | |
|---|---|---|---|---|---|
| HN derivertines | Sequences | IC50 | | Full protection | |
| HN | MAPRGFSCLLLLTSEIDLPVKRRA | 100 nM-1 | μM | ˜10 | μM* |
| HMG | MAPRGFSCLLLLTGEIDLPVKRRA | 100 pM-1 | nM | ˜10 | nM* |

| | | | |
|---|---|---|---|
| HNA | MAPRGFSALLLLTSEIDLPVKRRA | NE | NE* |

| | | | | | |
|---|---|---|---|---|---|
| P-S14 HN | MAPRGFSCLLLLT(p-S)EIDLPVKRRA | 100 nM-1 | μM | ˜10 | μM** |
| P-S7 HN | MAPRGF(p-S)CLLLLTSEIDLPVKRRA | 100 nM-1 | μM | ˜10 | μM* |
| P-S7/14 HN | MAPRGF(p-S)CLLLLT(p-S)EIDLPVKRRA | 100 nM-1 | μM | ˜10 | μM** |
| D-Ser14 HN | MAPRGFSCLLLLT(D-Ser)EIDLPVKRRA | 100 pM-1 | nM | ˜10 | nM* |
| D-Ser7 HN | MAPRGF(D-Ser)CLLLLTSEIDLPVKRRA | 100 nM-1 | μM | ˜10 | μM* |
| D-Ser7/14 HN | MAPRGF(D-Ser)CLLLLT(D-Ser)EIDLPVKRRA | 100 pM-1 | nM | ˜10 | nM* |
| AGA-(D-Ser14)HN | MAPAGASCLLLLT(D-Ser)EIDLPVKRRA | 10-100 | pM | 100-300 | pM* |
| AGA-(D-Ser14)HN17 | PAGASCLLLLT(D-Ser)EIDLP | 10-100 | pM | ˜100 | pM* |

| | | | |
|---|---|---|---|
| S7A HN | MAPRGFACLLLLTSEIDLPVKRRA | NE | NE* |

| | | | | | |
|---|---|---|---|---|---|
| S7A HNG17 | PRGFACLLLLTGEIDLP | 100 pM-1 | nM | ˜10 | nM** |

| | | | |
|---|---|---|---|
| HNG-KKK | YMAPRGFSCLLLLTGEIDLPVKKKK | NE | NE** |

| | | | | | |
|---|---|---|---|---|---|
| EF-(S7A)HN | EFLIVIKSMAPRGFACLLLLTSEIDLPVKRRA | 1-10 | nM | ˜100 | nM** |
| EF-HNG-KKK | EFLIVIKSMAPRGFSCLLLLTGEIDLPVKKKK | 100 pM-1 | nM | ˜10 | nM** |
| EF-HN | EFLIVIKSMAPRGFSCLLLLTSEIDLPVKRRA | 1-10 | nM | ˜100 | nM** |

| | | | |
|---|---|---|---|
| EF-HNA | EFLIVIKSMAPRGFSALLLLTSEIDLPVKRRA | NE | NE** |

| | | | | | |
|---|---|---|---|---|---|
| EF-HNG | EFLIVIKSMAPRGFSCLLLLTGEIDLPVKRRA | 10-100 | pM | ˜1 | nM* |

| | | | |
|---|---|---|---|
| EFLIVIKS | EFLIVIKS | NE | NE** |

| | | | | | |
|---|---|---|---|---|---|
| AGA-HNG | MAPAGASCLLLLTGEIDLPVKRRA | 10-100 | pM | 100-300 | pM* |
| EF-AGA-HNG | EFLIVIKSMAPAGASCLLLLTGEIDLPVKRRA | 100 fM-1 | pM | ˜10 | pM* |

The abbreviated names, the sequences, and the potencies of the suppressing actions are indicated for each peptide. The potency indicates the summary of the actions of each peptide against Aβ and four FAD genes (V642I-APP, NL-APP, M146L-PS1, and N141I-PS2), indicated as *, or against Aβ and V642I-APP, indicated as **. “Full protection” indicates the minimal concentration of each peptide that exerted full cytoprotection. “Full NE” indicates the corresponding peptide had no cytoprotective effect at 10 μM. p-S: phosphorylated L-Ser. Table 5 also includes the dimerization peptide, EFLIVIKS (SEQ ID NO: 20).

### INDUSTRIAL APPLICABILITY

The present invention provides Humanin derivatives that are useful in protecting neuronal cells from cytotoxicity related to neurodegenerative diseases. With higher potency and resulting lower effective amounts, HN derivatives represent better therapeutic agents than the naturally occurring form of HN. The lower effective amount of the peptides can minimize toxicity or side effects that might be associated with the HN derivatives. Also, the lower effective amount renders the manufacturing (e.g., genetic engineering or a chemical synthesis) and purifying of these peptides economically more practical. In addition, the HN derivatives provide the pharmaceutical industry an effective tool to study the pathological mechanism of neurodegenerative diseases and to develop more drugs.

### Other Embodiments

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

### REFERENCES

[1] Shastry, B. S. and Giblin, F. J. (1999) Brain. Res. Bull. 48, 121-127
[2] Yamatsuji, T., Matsui, T., Okamoto, T., Komatsuzaki, K., Takeda, S., Fukumoto, H., Iwatsubo, T., Suzuki, N., Asami-Odaka, A., Ireland, S., Kinane, T. B., Giambarella, U., and Nishimoto, I. (1996) Science 272, 1349-1352
[3] Wolozin, B., Iwasaki, K., Vito, P., Ganjei, J. K., Lacana, E., Sunderland, T., Zhao, B., Kusiak, J. W., Wasco, W., and D'Adamio, L. (1996) Science 274, 1710-1713
[4] Guo, Q., Furukawa, K., Sopher, B. L., Pham, D. G., Xie, J., Robinson, N., Martin, G. M., and Mattson, M. P. (1996) Neuroreport 8, 379-383
[5] Guo, Q., Sebastian, L., Sopher, B. L., Miller, M. W., Glazner, G. W., Ware, C. B., Martin, G. M., and Mattson, M. P. (1999) Proc. Natl. Acad. Sci. USA. 96, 4125-4130
[6] Zhao, B., Chrest, F. J., Horton, W. E. Jr., Sisodia, S. S., and Kusiak, J. W. (1997) J. Neurosci. Res. 47, 253-263
[7] Zhang, Z., Hartmann, H., Do, V. M., Abramowski, D., Sturchler-Pierrat, C., Staufenbiel, M., Sommer, B., van de Wetering, M., Clevers, H., Saftig, P., De Strooper, B., He, X., Yankner, B. A. (1998) Nature 395, 698-702
[8] Czech, C., Lesort, M., Tremp, G., Terro, F., Blanchard, V., Schombert, B., Carpentier, N., Dreisler, S., Bonici, B., Takashima, A., Moussaoui, S., Hugon, J., and Pradier, L. (1998) Neuroscience 87, 325-336
[9] Weihl, C. C., Ghadge, G. D., Kennedy, S. G., Hay, N., Miller, R. J., and Roos, R. P. (1999) J. Neurosci. 19, 5360-5369
[10] Luo, J. J., Wallace, W., Riccioni, T., Ingram, D. K., Roth, G. S., and Kusiak, J. W. (1999) J. Neurosci. Res. 55, 629-642
[11] Niikura, T., Murayama, N., Hashimoto, Y., Ito, Y., Yamagishi, Y., Matsuoka, M., Takeuchi, Y., Aiso, S., and Nishimoto, I. (2000) Biochem. Biophys. Res. Commun. 274,445-454
[12] Niikura, T., Hashimoto, Y., Okamoto, T., Abe, Y., Yasukawa, T., Kawasumi, M., Hiraki, T., Kita, Y., Terashita, K., Kouyama, K., and Nishinioto, I. (2001) J. Neurosci. 21, 1902-1910
[13] Hashimoto, Y., Niikura, T., Ito, Y., and Nishimoto, I. (2000) J. Biol. Chem. 275, 34541-34551
[14] Hashimoto, Y., Niikura, T., Tajima, H., Yasukawa, Y., Sudo, H., Ito, Y., Kita, Y., Kawasumi, M., Kouyama, K, Doyu, M., Sobue, G., Koide, T., Tsuji, S., Lang, J., Kurokawa, K., and Nishimoto, I. (2001) Proc. Natl. Acad. Sci. USA. 98,6336-6341
[15] Hashimoto, Y., Ito, Y., Niikura, T., Shao, Z., Hata, M., Oyama, F., and Nishimoto, I. (2001) Biochem. Biophys. Res. Commun. 283, 460-468
[16] Hashimoto, Y., Niikura, T., Ito, Y., Sudo, H., Hata, M., Arakawa, E., Abe, Y., Kita, Y., and Nishimoto, I. (2001) J. Neurosci. 21, 9235-9245
[17] Hashimoto, Y., Niikura, T., Ito, Y., Kita, Y., Terashita, K., and Nishimoto, I. (2002) J. Pharmacol. Exp. Ther. 300, 736-745
[18] Hashimoto, Y., Ito, Y., Arakawa, E., Kita, Y., Terashita, K., Niikura, T., and Nishimoto, I. (2002) J. Neurochem. 80, 426-437
[19] D'Adamio, L., Lacana, E., and Vito, P. (1997) Semin. Immunol. 9, 17-23
[20] Tajima, H., Niikura, T., Hashimoto, Y., Ito, Y., Kita, Y., Terashita, K., Yamazaki, K., Koto, A., Aiso, S., and Nishimoto, I. (2002) Neurosci. Lett. in press
[21] Yamatsuji, T., Okamoto, T., Takeda, S., Murayama, Y., Tanaka, N., and Nishimoto, I. (1996) EMBO. J. 15, 498-509.
[22] Platika, D., Boulos, M. H., Braizer, L., and Fishman, M. C. (1985) Proc. Natl. Acad. Sci. USA. 82, 3499-3503
[23] Hashimoto, Y., Jiang, H., Niikura, T., Ito, Y., Hagiwara, A., Umezawa, K., Abe, Y., Murayama, Y., and Nishimoto, I. (2000b) J. Neurosci. 20, 8401-8409
[24] Loo, D. T., Copani, A., Pike, C. J., Whittemore, E. R., Walencewicz, A. J., and Cotman, C. W. (1993) Proc. Natl. Acad. Sci. USA. 90, 7951-7955
[25] Gschwind, M., and Huber, G. (1995) J. Neurochem. 65, 292-300
[26] Sudo, H., Hashimoto, Y., Niikura, T., Shao, Z., Yasukawa, T., Ito, Y., Yamada, M., Hata, M., Hiraki, T., Kawasumi, M., Kouyama, K., and Nishimoto, I. (2001) Biochem. Biophys. Res. Commun. 282, 548-556
[27] Storms, S. D., and Rutishauser, U. (1998) J. Biol. Chem. 273, 27124-27129
[28] Huang, P., Miao, S., Fan, H., Sheng, Q., Yan, Y., Wang, L., and Koide, S. S. (2000) Mol. Hum. Reprod. 6, 1069-1078
[29] Sudo, H., Jiang, H., Yasukawa, T., Hashimoto, Y., Niikura, T., Kawasumi, M., Matsuda, S., Takeuchi, Y., Aiso, S., Matsuoka, M., Murayama, Y., and Nishimoto, I. (2000) Mol. Cell. Neurosci. 16, 708-723
[30] Hagiwara, A., Hashimoto, Y., Niikura, T., Ito, Y., Terashita, K., Kita, Y., Nishimoto, I., and Umezawa, K. (2000) Biochem. Biophys. Res. Commun. 278, 633-639
[31] Ghil, S. H., Kim, B. J., Lee, Y. D., and Suh-Kim, H. (2000) J. Neurochem. 74, 151-158
[32] Gururaja, T. L., Narasimhamurthy, S., Payan, D. G., and Anderson, D. C. (2000) Chem. Biol. 7, 515-527
[33] Xu, X., Leo, C., Jang, Y., Chan, E., Padilla, D., Huang, B. C., Lin, T., Gururaja, T., Hitoshi, Y., Lorens, J. B., Anderson, D. C., Sikic, B., Luo, Y., Payan, D. G., and Nolan, G. P. (2001) Nat. Genet. 27, 23-29
[34] Wolosker, H., Blackshaw, S., and Snyder, S. H. (1999) Proc. Natl. Acad. Sci. USA. 96, 13409-13414

1. A derivative of Humanin that protects a neuronal cell from cytotoxicity, wherein the derivative contains at least one D-amino acid or phosphorylated amino acid.

2. The derivative of claim 1, wherein the position of the D-amino acid corresponds to the position of ¹⁴Ser of Humanin.

3. The derivative of claim 1, wherein the D-amino acid is a D-Serine.

4. The derivative of claim 1, wherein the D-amino acid is a D-Proline.

5. A derivative of Humanin that protects a neuronal cell from cytotoxicity, wherein the derivative has an addition of one or more amino acids having an activity of forming a multimer.

6. The derivative of claim 5, wherein the derivative contains one or more amino acids having an activity of binding to each other.

7. The derivative of claim 6, wherein the sequence of the amino acids contains EFLIVIKS or EFLIVKS.

8. The derivative of claim 1, wherein the derivative is selected from the group consisting of polypeptides defined as: “HN with S14P”, “(D-Ser¹⁴)HN”, “(D-Ser⁷)HN”, “(D-Ser^{7/14})HN”, “AGA-(D-Ser¹⁴)HN , “AGA-(D-Ser¹⁴)HN17”, “EFLIVIKS-HNG”, “EFLIVIKS-HNG-KKK”, “EFLIVIKS-(S7A)HN”, “EFLIVIKS-AGA-HNG”, and chimeric combinations thereof.

9. A nucleic acid encoding the derivative of claim 1.

10. A method for protecting a neuronal cell from cytotoxicity, wherein the method comprises the step of contacting the derivative of claim 1 to the cell.

11. A pharmaceutical composition comprising the derivative of claim 1, and one or more pharmaceutically acceptable carriers.

12. A method for treating an individual that is suffering from or suspected of having a neurodegenerative disorder, wherein the method comprises the step of administrating the derivative of claim 1 to the individual.

13. The method of claim 12, wherein the disorder is Alzheimer's disease.

14. The derivative of claim 5, wherein the derivative is selected from the group consisting of polypeptides defined as: “HN with S14P”, “(D-Ser14)HN”, “(D-Ser7)HN”, “(D-Ser7/14)HN”, “AGA-(D-Ser14)HN”, “AGA-(D-Ser14)HN17”, “EFLIVIKS-HNG”, “EFLIVIKS-HNG-KKK”, “EFLIVIKS-(S7A)HN”, “EFLIVIKS-AGA-HNG”, and chimeric combinations thereof.

15. A nucleic acid encoding the derivative of claim 5.

16. A method for protecting a neuronal cell from cytotoxicity, wherein the method comprises the step of contacting the derivative of claim 5 to the cell.

17. A pharmaceutical composition comprising the derivative of claim 5, and one or more pharmaceutically acceptable carriers.

18. A method for treating an individual that is suffering from or suspected of having a neurodegenerative disorder, wherein the method comprises the step of administrating the derivative of claim 5 to the individual.
